(19) United States
(12) Patent Application Publication
(10) Pub. No.: US 2009/0247466 A1
(43) Pub. Date: Oct. 1, 2009

(54) NEUROPROTECTIVE INTEGRIN-BINDING PEPTIDE AND ANGIOPOIETIN-1 TREATMENTS

(75) Inventors: Theodoor Hagg, Louisville, KY (US); Scott R. Whittemore, Louisville, KY (US)

Correspondence Address: VIKSNINS HARRIS & PADYS PLLP, P.O. BOX 111098, ST. PAUL, MN, 55111-1098, US

(73) Assignee: University of Louisville Research Foundation, Louisville, KY (US)

(21) Appl. No.: 12/401,393

(22) Filed: Mar. 10, 2009

Related U.S. Application Data
(60) Provisional application No. 61/035,308, filed on Mar. 10, 2008.
(60) Provisional application No. 61/049,969, filed on May 2, 2008.

(51) Int. Cl. A61K 38/07; A61K 38/08; A61P 25/28; A61P 27/02; A61K 38/10

(52) U.S. Cl. 514/14; 514/18

(57) ABSTRACT

The present invention provides therapeutic compositions, kits and methods for treating nervous system injury in a mammal. Certain methods include administering to a mammal in need of such therapy an effective amount of angiopoietin-1 (Ang-1), or a functional analog thereof. Certain methods include administering a peptide having 4 to 20 amino acids that comprises an YVRL (SEQ ID NO:1) motif that may have activity at the αvβ3 or α5β1 integrin receptor, such as C-16, or a compound that is an agonist at the αvβ3 and/or α5β1 integrin receptor, which administration may be in combination with administration of Ang-1, or a functional analog thereof.

### RELATED APPLICATIONS

This application claims the benefit of priority of U.S. application Ser. No. 61/035,308, filed Mar. 10, 2008, and of U.S. application Ser. No. 61/049,969, filed May 2, 2008, which applications are herein incorporated by reference.

### STATEMENT OF GOVERNMENT SUPPORT

This invention was made with government support under grant numbers NS45734 and RR015576 awarded by the National Institutes of Health. The government has certain rights in the invention.

### BACKGROUND

Protection of the motor and sensory systems and the spinal cord circuitries in mammals during the acute and sub-acute phase of a spinal cord injury, prior to or during surgery, or to treat a degenerative disease, would lead to great improvement in the quality of life, such as movement, touch, appropriate pain responses, and control of various bodily functions.

Currently, there is a need for neuroprotective treatments.

### SUMMARY OF CERTAIN EMBODIMENTS OF THE INVENTION

Certain embodiments of the present invention provide compounds and compositions that are neuroprotective and/or anti-inflammatory. Accordingly, the present invention provides a therapeutic method for treating neural injury (e.g., traumatic neural injury) or a degenerative disorder in a mammal (such as a human male or female, a cat, a dog, a horse, a donkey, a mule, a cow, a sheep, a goat, a camel, etc.) comprising administering to a mammal in need of such therapy an effective amount of a therapeutic compound that has activity at the αvβ3 or α5β1 integrin receptor.

Certain embodiments of the present invention provide a therapeutic method for treating nervous system injury in a mammal by administering to the mammal in need of such therapy an effective amount of a therapeutic compound, wherein the therapeutic compound is a 4 to 20 amino acid peptide comprising an YVRL (SEQ ID NO:1) motif that has activity at the αvβ3 or α5β1 integrin receptor.

Certain embodiments of the present invention provide a therapeutic method for pre-treating a mammal, such as a human, prior to surgery to prevent injury to nerves. In certain embodiments, the method involves administering to a mammal in need of such therapy an effective amount of a therapeutic compound, wherein the therapeutic compound is a 4 to 20 amino acid peptide comprising an YVRL (SEQ ID NO:1) motif that has activity at the αvβ3 or α5β1 integrin receptor.

Certain embodiments of the present invention provide a therapeutic method for rescuing blood vessels in a mammal by administering to the mammal in need of such therapy an effective amount of a therapeutic compound, wherein the therapeutic compound is a 4 to 20 amino acid peptide comprising an YVRL (SEQ ID NO:1) motif that has activity at the αvβ3 or α5β1 integrin receptor.

Certain embodiments of the present invention provide a therapeutic method for reducing transmigration of leukocytes (e.g., monocytes) across endothelia in a mammal by administering to the mammal in need of such therapy an effective amount of therapeutic compound, wherein the a therapeutic compound is a 4 to 20 amino acid peptide comprising a YVRL (SEQ ID NO:1) motif that has activity at the αvβ3 or α5β1 integrin receptor.

Certain embodiments of the present invention provide a therapeutic method for stimulating angiogenesis in a mammal by administering to the mammal in need of such therapy an effective amount of a therapeutic compound, wherein the therapeutic compound is a 4 to 20 amino acid peptide comprising an YVRL (SEQ ID NO:1) motif that has activity at the αvβ3 or α5β1 integrin receptor.

Certain embodiments of the present invention provide a therapeutic method for treating an inflammatory condition in a mammal by administering to the mammal in need of such therapy an effective amount of a therapeutic compound, wherein the therapeutic compound is a 4 to 20 amino acid peptide comprising an YVRL (SEQ ID NO:1) motif that has activity at the αvβ3 or α5β1 integrin receptor.

Certain embodiments of the present invention provide a therapeutic method for treating a degenerative disorder in a mammal, such as a human, by administering to a mammal in need of such therapy an effective amount of a therapeutic compound, wherein the therapeutic compound is a 4 to 20 amino acid peptide comprising a YVRL (SEQ ID NO:1) motif that has activity at the αvβ3 or α5β1 integrin receptor.

Certain embodiments of the present invention provide a therapeutic method for treating neural injury (e.g., traumatic neural injury) or a degenerative disorder in a mammal (such as a human male or female, a cat, a dog, a horse, a donkey, a mule, a cow, a sheep, a goat, a camel, etc.) comprising administering to a mammal in need of such therapy an effective amount of a neuroprotective compound, wherein the neuroprotective compound is C16.

In certain embodiments, the neuroprotective compound is administered directly into and/or around the injured tissue, is administered through delivery into the cerebrospinal fluid, or is administered intravenously.

In certain embodiments, the neural injury (e.g., traumatic neural injury) is a spinal cord injury, a brain injury, a peripheral nerve injury, an eye injury affecting the optic nerve fibers, or a skin burn. In certain embodiments, the neural injury (e.g., traumatic neural injury) is caused by an ischemic or hemorrhagic stroke. In certain embodiments, the degenerative disorder is a neuron, axon, or myelin disorder, or an oligodendrocyte disorder, such as multiple sclerosis or peripheral neuropathy. In certain embodiments the disorder is caused by blood vessel dysfunction. In certain embodiments, the neuroprotective compound is administered as a pre-treatment prior to surgery.

Certain embodiments of the present invention provide a neuroprotective compound as described herein for the manufacture of a medicament useful for the treatment of a neural injury (e.g., traumatic neural injury) or a degenerative disorder in a mammal, wherein the neuroprotective compound is C16.

Certain embodiments of the present invention provide a therapeutic method for treating an inflammatory condition in a mammal comprising administering to the mammal in need of such therapy an effective amount of an anti-inflammatory compound, wherein the anti-inflammatory compound is C16.

Certain embodiments of the present invention provide the use of a therapeutic compound for the manufacture of a medicament useful for the treatment of a nervous system injury, for pre-treating a mammal, such as a human, prior to surgery to prevent injury to nerves, for rescuing blood vessels, for reducing transmigration of monocytes across endothelia, for stimulating angiogenesis, for treating an inflammatory condition, or for treating a degenerative disorder, wherein the therapeutic compound is a 4 to 20 amino acid peptide comprising a YVRL (SEQ ID NO:1) motif that has activity at the αvβ3 and/or α5β1 integrin receptor.

Certain embodiments of the present invention provide the use of a therapeutic compound for the manufacture of a medicament useful for the treatment of a nervous system injury, for pre-treating a mammal, such as a human, prior to surgery to prevent injury to nerves, for rescuing blood vessels, for reducing transmigration of monocytes across endothelia, for stimulating angiogenesis, for treating an inflammatory condition, or for treating a degenerative disorder, wherein the therapeutic compound is C16.

Certain embodiments of the present invention provide nucleic acids that encode the peptides described herein.

Described herein are experiments that demonstrate that intravenous treatment with an Ang-1 mimetic, with or without the C16 peptide, provides permanent protection for myelin and function after a traumatic spinal cord injury. Such treatments reduce detrimental inflammation (number of leukocytes and microglial activation) within the injured spinal cord area. Treatment with the combination of Ang-1 and C16 appears to be better than either Ang-1 or C16 alone. Ang-1 activates the Tie2 receptor and C16 binds to αvβ3 and α5β1 integrin receptors.

Thus, certain embodiments of the present invention provide compounds and compositions that are neuroprotective. Certain embodiments of the present invention provide compounds and compositions that are anti-inflammatory.

Accordingly, the present invention provides a therapeutic method for treating neural injury (e.g., traumatic neural injury) or a degenerative disorder in a mammal (such as a human male or female, a cat, a dog, a horse, a donkey, a mule, a cow, a sheep, a goat, a camel, etc.).

Certain embodiments of the present invention provide therapeutic methods for treating nervous system injury in a mammal comprising administering to the mammal in need of such therapy an effective amount of a first therapeutic compound, wherein the first therapeutic compound is angiopoietin-1 (Ang-1), or a functional analog thereof.

Certain embodiments of the present invention provide therapeutic methods for pre-treating a mammal, such as a human, prior to surgery to prevent injury to nerves, comprising administering to the mammal in need of such therapy an effective amount of a first therapeutic compound, wherein the first therapeutic compound is angiopoietin-1 (Ang-1), or a functional analog thereof.

Certain embodiments of the present invention provide therapeutic methods for rescuing blood vessels in a mammal comprising administering to the mammal in need of such therapy an effective amount of a first therapeutic compound, wherein the first therapeutic compound is angiopoietin-1 (Ang-1), or a functional analog thereof.

Certain embodiments of the present invention provide therapeutic methods for reducing transmigration of leukocytes across endothelia in a mammal comprising administering to the mammal in need of such therapy an effective amount of a first therapeutic compound, wherein the first therapeutic compound is angiopoietin-1 (Ang-1), or a functional analog thereof.

Certain embodiments of the present invention provide therapeutic methods for stimulating angiogenesis in a mammal comprising administering to the mammal in need of such therapy an effective amount of a first therapeutic compound, wherein the first therapeutic compound is angiopoietin-1 (Ang-1), or a functional analog thereof.

Certain embodiments of the present invention provide therapeutic methods for treating an inflammatory condition, such as uveitis or Alzheimer's disease, in a mammal comprising administering to the mammal in need of such therapy an effective amount of a first therapeutic compound, wherein the first therapeutic compound is angiopoietin-1 (Ang-1), or a functional analog thereof.

Certain embodiments of the present invention provide therapeutic methods for treating a degenerative disorder in a mammal, comprising administering to the mammal in need of such therapy an effective amount of a first therapeutic compound, wherein the first therapeutic compound is angiopoietin-1 (Ang-1), or a functional analog thereof.

In certain embodiments of the invention, the methods may further comprise administering to the mammal a second therapeutic compound, wherein the second therapeutic compound is a 4 to 20 amino acid peptide comprising an YVRL (SEQ ID NO:1) motif.

In certain embodiments of the invention, the second therapeutic compound is the C16 peptide (KAFDITYVRLKF (SEQ ID NO:2)).

In certain embodiments of the invention, the second therapeutic compound has activity (e.g., is an agonist) at the αvβ3 or α5β1 integrin receptor.

In certain embodiments, the second therapeutic compound is a functional analog of C16 that does not comprise YVRL (SEQ ID NO:1). Such a compound may be an agonist at the αvβ3 and/or α5β1 receptor.

In certain embodiments of the invention, the first or first and second therapeutic compounds are administered directly into or around the injured tissue, administered through delivery into the cerebrospinal fluid, administered onto or directly into the eye or administered intravenously.

In certain embodiments of the invention, the degenerative disorder is a neuron, axon, or myelin disorder.

In certain embodiments of the invention, the degenerative disorder is an oligodendrocyte disorder.

In certain embodiments of the invention, the degenerative disorder is multiple sclerosis or peripheral neuropathy.

In certain embodiments of the invention, the first or first and second therapeutic compounds are administered directly into or around an injured tissue, administered through delivery into the cerebrospinal fluid, or administered intravenously.

In certain embodiments of the invention, the first or first and second therapeutic compounds are each administered at a concentration of less than 100 mg/kg/day.

In certain embodiments of the invention, the first or first and second therapeutic compounds are each administered at a concentration of between 1 ng/kg/day to 30 mg/kg/day.

In certain embodiments of the invention, the first or first and second therapeutic compounds are each administered at a concentration of between 1 mg/kg/day to 10 mg/kg/day.

In certain embodiments of the invention, the first or first and second therapeutic compounds are each administered at a concentration of between 3 mg/kg/day to 10 mg/kg/day.

In certain embodiments of the invention, the first or first and second therapeutic compounds are each administered at a concentration of between 1 mg/kg/day to 3 mg/kg/day.

In certain embodiments of the invention, the mammal is a male or female human, cat, dog, horse, donkey, mule, cow, sheep, goat, or camel.

In certain embodiments of the invention, the first or first and second therapeutic compounds are each administered for a period of about one to four weeks.

In certain embodiments of the invention, the nervous system injury is a traumatic nervous system injury.

In certain embodiments of the invention, the traumatic nervous system injury is a spinal cord injury, brain injury, or a peripheral nerve injury, an eye injury affecting the optic nerve fibers, or a skin burn.

In certain embodiments of the invention, the traumatic nervous system injury is a spinal cord injury.

In certain embodiments of the invention, the traumatic nervous system injury is caused by an ischemic or hemorrhagic stroke.

In certain embodiments of the invention, the first or first and second therapeutic compounds are each administered within about 0-48 hours of injury.

In certain embodiments of the invention, the first or first and second therapeutic compounds are each administered within about 0-24 hours of injury.

In certain embodiments of the invention, the first or first and second therapeutic compounds are each administered within about 0-12 hours of injury.

In certain embodiments of the invention, the first or first and second therapeutic compounds are each administered within about 0-5 hours of injury.

In certain embodiments of the invention, the second therapeutic compound is an agonist at the αvβ3 and/or α5β1 integrin receptor.

Certain embodiments of the present invention provide the use of angiopoietin-1 (Ang-1), or a functional analog thereof, for the manufacture of a medicament useful for the treatment of a nervous system injury, for pre-treating a mammal, prior to surgery to prevent injury to nerves, for rescuing blood vessels, for reducing transmigration of leukocytes across endothelia, for stimulating angiogenesis, for treating an inflammatory condition, or for treating a degenerative disorder.

Certain embodiments of the present invention provide the use of a first and second therapeutic compound for the manufacture of a medicament useful for the treatment of a nervous system injury, for pre-treating a mammal, prior to surgery to prevent injury to nerves, for rescuing blood vessels, for reducing transmigration of leukocytes across endothelia, for stimulating angiogenesis, for treating an inflammatory condition, or for treating a degenerative disorder, wherein the first therapeutic compound is angiopoietin-1 (Ang-1), or a functional analog thereof, and the second therapeutic compound is an agonist at the αvβ3 and/or α5β1 integrin receptor.

Certain embodiments of the present invention provide the use of a first and second therapeutic compound for the manufacture of a medicament useful for the treatment of a nervous system injury, for pre-treating a mammal, prior to surgery to prevent injury to nerves, for rescuing blood vessels, for reducing transmigration of leukocytes across endothelia, for stimulating angiogenesis, for treating an inflammatory condition, or for treating a degenerative disorder, wherein the first therapeutic compound is angiopoietin-1 (Ang-1), or a functional analog thereof, and the second therapeutic compound is a peptide having 4 to 20 amino acids comprising an YVRL (SEQ ID NO:1) motif.

Certain embodiments of the present invention provide pharmaceutical compositions comprising a first and second therapeutic compound, wherein the first therapeutic compound is angiopoietin-1 (Ang-1), or a functional analog thereof and the second therapeutic compound is an agonist at the αvβ3 and/or α5β1 integrin receptor, and a pharmaceutically acceptable carrier.

Certain embodiments of the present invention provide pharmaceutical compositions comprising a first and second therapeutic compound, wherein the first therapeutic compound is angiopoietin-1 (Ang-1), or a functional analog thereof and the second therapeutic compound is a peptide having 4 to 20 amino acids comprising an YVRL (SEQ ID NO:1) motif.

Certain embodiments of the present invention provide compositions as described herein for use in medical treatment or diagnosis.

Certain embodiments of the present invention provide kits comprising a first and second therapeutic compound, wherein the first therapeutic compound is angiopoietin-1 (Ang-1), or a functional analog thereof and the second therapeutic compound is an agonist at the αvβ3 and/or α5β1 integrin receptor.

Certain embodiments of the present invention provide kits comprising a first and second therapeutic compound, wherein the first therapeutic compound is angiopoietin-1 (Ang-1), or a functional analog thereof and the second therapeutic compound is a peptide having 4 to 20 amino acids comprising an YVRL (SEQ ID NO:1) motif.

### BRIEF DESCRIPTION OF THE FIGURES

FIG. 1. C16 reduces the volume of tissue loss 7 days after spinal cord contusion in adult mice. The total volume of the tissue damage was reduced.

FIG. 2A-2F. C16 reduces white matter loss and functional deficits 7 day after a spinal cord contusion. A) A transverse section of a sham-operated (laminectomy only) mouse shows white matter stained with eriochrome cyanine. B) White matter loss was extensive in mice injected intravenously with vehicle once daily over 7 days. C) C16 injections improved the white matter sparing. D) The total area of white matter was clearly greater at the injury epicenter in C16 treated mice than in those injected with vehicle or a control peptide SP3. The difference was not significant at 1 mm rostral or caudal from the injury epicenter. E) Overground locomotor function was assessed by Basso Mouse Scale (BMS) and showed a clear protective effect of C16 which correlated to the extent of white matter sparing (F). The regression analysis was performed on the mice mice from the 3 groups combined.

FIG. 3. A dose-response study showed that 100 microgram C16 per day was the lowest maximally effective dose in terms of white matter sparing at the injury epicenter 7 days after injury.

FIG. 4A-4F. C16 treatment provides lasting neuroprotection. A) C16 treatment over the first 14 days and started 4 hours after a contusion injury at spine level T9 improved the BMS score as early as 7 days after injury and well beyond the termination of the treatment. B) Transverse sections through the epicenter shows extensive loss of white matter in vehicle treated mice 6 weeks after injury, which was improved in mice treated with C16 (C). D) A comparison between the white matter sparing at 7 days and 42 days shows that the neuroprotective effects of C16 group were permanent. E) A regression analysis of the individual mice shows that white matter sparing at 42 days correlates well with the BMS scores at 42 days as well as at 7 days (F). The latter observation again suggests that most neuroprotective effects of C16 are during the first week following injury. The regression analysis was performed on the mice from the 2 groups combined.

FIG. 5A-5B. A bolus injection of C16 is also neuroprotective. A) A single injection of 100 microgram C16 immediately after the injury also reduces the functional deficits (A) and white matter sparing (B) at 7 days following a T9 contusion.

FIG. 6A-6E. C16 rescues blood vessels and induces angiogenesis. A) A time course of the number of blood vessels in the injury penumbra shows a rapid loss of blood vessels in vehicle treated mice 24 hours after a spinal cord contusion at T9 which remains low over 42 days. Perfused blood vessels were identified by intravenous injection of LEA lectin 30 minutes before analysis. A single injection of C16 reduces the loss seen after 24 hours. A 7 day treatment increases the number of vessels, suggesting that C16 causes angiogenesis. At 7 days, the number of LEA labeled blood vessels correlated with locomotor performance (B; BMS), white matter at the epicenter (C). However, at 42 days the blood vessel number do not predict the BMS (D) or white matter sparing (E).

FIG. 7A-7H. C16 reduces inflammation after spinal cord injury. A) A time course shows that C16 treated mice have fewer CD45 positive infiltrating leukocytes in a 6 mm segment of the spinal cord around the epicenter at all post-injury time points analyzed. The area is the sum of areas at each of the 1 mm distances within the segment (3 mm rostral to 3 mm caudal from the epicenter). B) CD68 is a marker for activated resident microglia and macrophages and shows a more extensive area than CD45 in both C16 and vehicle treated mice. C16 also reduces the area of CD68 staining at all time points following injury. At 7 days, the rostrocaudal distribution of the CD45-positive (C) and CD68-positive (D) area shows effects throughout the injury site and beyond. Regression analyses show a relationship between the inflammation as assessed by CD45 and white matter sparing (E) and locomotor function (F). Circles=vehicle, triangles=SP3 control peptide, filled squares=C16. The regression analysis was performed on the mice from the 3 groups combined. At 42 days, CD45 correlated well with the white matter area at the epicenter (G) and the BMS (H), suggesting that chronic inflammation contributes to dysfunction.

FIG. 8A-8B. C16 reduces monocyte transmigration across endothelial cells in vitro. A) The number of monocytic cells (THP-1 cells) that had migrated across a monolayer of endothelial cells to a separate compartment in culture wells was reduced by 400 and 600 μM C16 in the absence or presence of the pro-inflammatory cytokine TNFα. SP3 control peptide had a similar level of transmigration as in controls without the peptide (not shown). B) Transmigration was avb3 dependent as shown by blocking antibodies in the presence of TNFα. The α5β1 integrin, which can also bind C16, was not involved, as α5 antibodies failed to affect transmigration. The extent of reduced transmigration was much less with the antibody than with C16.

FIG. 9. FIG. 9A. Mice received a contusion at T9 and were injected intravenously with vehicle, Ang-1TFD (Ang-1) or Ang-1TFD plus C16 for 7 days. Behavioral analysis was performed once a week using the standard BMS scoring method for overground locomotor function. A score of 5 and above indicates hind-limb weight support and plantar stepping, whereas a score of 7 and above is highly functional. The BMS scores of the two Ang-1-containing treatment groups were significantly higher at all post-injury times and lasted well beyond the 1 week treatment period. Baseline=score in the week before the surgery. Overall, the combination treatment was not significantly better than Ang-1 alone (p=0.092) but was better than the Ang-1 treatment at 8 and 15 days (*; p<0.05). However, at 43 days 6/10 mice with the Ang-1+C16 treatment had a score of 7 or above compared to 4/14 in the Ang-1 group and none in the vehicle group. A previous study showed an end-point BMS of 5.5 with C16 alone. Thus, the combination of Ang-1TFD plus C16 provides better functional outcomes than either agent alone. Vehicle n=13, Ang-1 n=14, Ang-1+C16 n=10. Statistical analyses were performed by Two Way Repeated Measures ANOVA (One Factor Repetition) followed by Student-Newman-Keuls Method.

FIG. 9B. FIG. 9B depicts the percentage of mice in different functional categories of the hind-limb BMS scores seen in the last two weeks of testing, i.e., 5 and 6 weeks after injury. Scores of 0-2 represent paralyzed or severely impaired, 3-4 represent impaired with some ankle movement, 5-6 represent moderately impaired with some coordination of stepping, 7-8 represent consistent stepping and coordination with trunk instability and 9 represent perfect and not distinguishable from normal (Basso et al., J Neurotrauma, 23(5), 635-659 (2006)). This provides additional evidence that a combination of Ang-1TFD plus C16 provides better functional outcomes than Ang-1TFD or C16 alone.

FIG. 10. After 6 weeks, the mice in the first group were processed for histology, showing improved white matter sparing at the injury epicenter (p<0.05, 0.0005 vs. veh) and reduced CD45+infiltrated leukocytes in the spinal cord segment 3 mm rostral to caudal from the injury (p<0.05, 0.005 vs. veh). The white matter sparing was not significantly different between the Ang-1 and Ang-1+C16 treatments. CD45 values were lower after the Ang-1+C16 treatment than Ang-1 alone (p<0.05). Vehicle n=7, Ang-1 n=7, Ang-1+C16 n=4.

FIG. 11. Mice received a contusion and were injected intravenously with vehicle or reagents for 7 days and processed for histology. All treatments show improved white matter sparing at the injury epicenter (p<0.05, 0.005, 0.005 vs. veh, respectively) and reduced CD45, a marker of infiltrated leukocytes (p<0.005, 0.01, 0.05, respectively). FIG. 11C shows the distribution of CD45 at rostral (R) and caudal (C) 1 mm distances from the epicenter. (n=5,4,4,5).

FIG. 12. Mice received a contusion and were injected intravenously with vehicle, C16 or Ang-1 once immediately after the injury. 24 hours later they received an intravenous LEA lectin injection to label perfused blood vessels and were processed for histology. The left panel shows an example in a small region of a cross section of the spinal cord. LEA+vessels were counted in the penumbra of the injury site. Both C16 and Ang-1 treatments show improved blood vessel sparing (p<0.05, 0.005, vs. veh) and reduced CD45, a marker of infiltrated leukocytes (p<0.05, 0.05 vs. veh). n=5 each.

FIG. 13. An i.v. C16 plus Ang-1 treatment provides superior and lasting improvement in locomotor function following SCI in mice. A) Daily i.v. injections with C16 over 14 days (solid squares, n=9) reduce locomotor deficits (measured by BMS), following a T9 contusion in mice compared to vehicle control injections (open squares, n=8). The benefit lasted beyond termination of the treatment and mice reached a score of 5 (horizontal line), indicating weight bearing and stepping. Mice analyzed for histology at 7 days show that C16 (open triangle, n=8) also causes better outcomes compared to vehicle (closed circle, n=13) or SP3 peptide (open circle, n=8) controls. B) Daily i.v. injections over 7 days with Ang-1 (solid triangle, n=14) or Ang-1 plus C16 (open diamonds, n=10) greatly improve BMS scores compared to vehicle (open squares, n=13). C) The percentage of mice in different categories of hind-limb function seen over the last two weeks of testing. The vehicle groups from (A) and (B) were not statistically different and were combined. BMS scores of 0-2: paralyzed, 3-4: some ankle movement, 5-6: some coordination and stepping, 7-8: consistent coordinated stepping with trunk instability, 9: normal. D) Averages of scores of 5 and over during the last two weeks are higher with the combination treatment than with C16, Ang-1 or vehicle alone. Four of the vehicle mice had a score of 5 and over. * p<0.05; ** p<0.01; *** p<0.001 compared to vehicle or as indicated by lines.

Figure. C16 and Ang-1 treatments reduce white matter loss following SCI. Compared to a sham-operated (laminectomy only) mouse with normal white matter (A), a mouse mice injected i.v. with vehicle over 7 days (B) has extensive loss of white matter as shown by myelin staining with eriochrome cyanine in transverse sections at the injury epicenter. C) Injections of C16 (shown here) or Ang-1 or their combination increased the amount of spared white matter. D) The total area of white matter at the injury epicenter (as a percentage of sham) shows that C16, Ang-1, and C16 plus Ang-1 treatments improve white matter sparing compared to vehicle or SP3 controls at 7 and 42 days post-injury. A single bolus of C16 given immediately following the injury (1× C16) also results in white matter sparing seen at 7 days post-injury. Scale bar in (A) is 200 μm. Data are mean±SEM. Group numbers are indicated in the bars. * p<0.05; ** p<0.01; *** p<0.001 compared to vehicle.

FIG. 15. C16 and Ang-1 treatments rescue blood vessels after SCI. To evaluate the extent of rescue of perfused blood vessels, LEA was injected intravenously 30 minutes before histological processing. A) LEA-labeled blood vessels in sham-operated mice had a normal appearance. The box in the inset schematic represents the region presented in A-C. B) 7 days following SCI, mice treated for 7 days with vehicle show few blood vessels, whereas C) C16-treated mice have many more. D) A time course shows a reduction in the number of blood vessels in the injury penumbra in vehicle-treated mice by 24 hours following SCI. Single injections of C16 or Ang-1 rescues blood vessels after 24 hours and with 7 day injections the numbers remain higher than with vehicle. The 7 day treatment with C16 increases the number of vessels compared to 24 hours. At both 7 (E,F) and 42 days (G,H), the number of LEA-labeled blood vessels correlated with locomotor performance as measured by BMS (E,G) and spared white matter at the epicenter (F,H). Data are mean±SEM. Sham and normal mice, n=8; 1 day vehicle, n=5; 1 day C16, n 5; 1 day Ang-1, n=5; 7 day vehicle, n=13; 7 day C16, n=8; 7 day Ang-1, n=5; 6 week vehicle, n=22; 6 week C16, n=12; 6 week Ang-1, n=7, C16+Ang-1, n=6. The numbers of mice are not the same as in FIGS. 13 and 14 as not all mice received LEA injections and not all mice with LEA injections were tested for BMS. Scale bar in (C) is 100 μm. * p<0.05; ** p<0.01; *** p<0.001 compared to vehicle or as indicated by the vertical line at 7 days.

FIG. 16. C16 and Ang-1 treatments reduce inflammation after SCI. A) A transverse section at the injury epicenter stained for CD45 shows extensive infiltration of leukocytes at 7 days post-injury in a mouse injected i.v. with vehicle over the same period. B) Injections of C16 (shown here) or Ang-1 or their combination greatly reduced the infiltration. C and D) CD68, a marker for activated microglia and macrophages, was similarly reduced by C16. E) The total cross-sectional area of CD45-positive cells at the injury site shows that C16, Ang-1 or C16 plus Ang-1 treatments reduce infiltration compared to vehicle or SP3 controls at 7 and 42 days post-injury but not significantly at 24 hours. The area is the sum of areas at 1 mm distances within a segment from 3 mm rostral to 3 mm caudal to the epicenter and is shown as a percentage of the vehicle group within the experiment. F) The cross-sectional area of CD68-positive cells at the injury site shows that C16, Ang-1 and C16 plus Ang-1 treatments reduce microglia/macrophage activation at all post-injury times. Scale bar in (D) is 200 μm and in the higher magnification insets, 50 μm. Data are mean±SEM. Group numbers are as in FIG. 14 plus n=5 each at 24 hr. * p<0.05; ** p<0.01; *** p<0.001 compared to vehicle or as indicated by the horizontal line.

FIG. 17. C16 reduces monocyte transmigration across ECs in vitro. A) The number of monocytes (THP-1 cells) that had migrated across a monolayer of ECs to a separate compartment in transwells was reduced by 600 μM C16 in the absence or presence of the pro-inflammatory cytokine TNFα. SP3 control peptide had no significant effect. Values are expressed as a percentage of vehicle (+SEM). B) Transmigration was αvβ3 dependent as shown by blocking antibodies in the presence of TNFα. The α5β1 integrin, which can also bind C16, was not involved, as α5 antibodies failed to affect transmigration. The extent of reduced transmigration was much less with the antibody than with C16. Data are mean±SEM, n=3 each. *p<0.05; **p<0.01; ***p<0.001 compared to vehicle or control.

FIG. 18. αvβ3 integrin is present on blood vessels after SCI. Twenty-four hours after a contusive SCI, immunostaining for αv (A), β3 (D) or α5β1 (G) integrin is seen at the epicenter on blood vessels identified by i.v. injection of LEA (B,E,H). The XZ and YZ views of these confocal images (C,F,I) confirm the co-localization of LEA and the integrins. Some neurons also stain for the integrins (arrows) which disappeared at the injury epicenter (not shown). Three days post-injury, spinal cord sections through the epicenter show αv (J), β3 (K) or α5β1 (L) integrin staining in some neurons (arrows) and a few blood vessels, but staining for both αv and β3 or α5β1 integrin is not seen in many other cells, including the numerous inflammatory cells expected in these injured tissues. Scale bars are indicated.

FIG. 19. C16 reduces the volume of tissue loss 7 days after SCI. A) Horizontal section shows a heterodomain characterized by deposits of laminin by invading mesenchymal cells which replace lost spinal cord tissue in a mouse contused 7 days before at T9. B) C16 treatment over 7 days reduced the size of the heterodomains. With vehicle treatment, tissue loss occurred over half the diameter of the spinal cord, whereas with C16 treatment damage appeared to be less in the outer regions of the spinal cord, including white matter tracts. Note the preservation of blood vessels identified by laminin-positive basement membrane in the injury penumbra. C) The total volume of the lamininpositive heterodomains showed a 43% reduction after C16 treatment compared to vehicle treatment. Data are mean±SEM; vehicle n=10, C16 n=11. ** p<0.01.

FIG. 20. C16 is effective in treating uveitis.

### DETAILED DESCRIPTION

Spinal cord injury results in loss of function and progressive secondary tissue degeneration, leaving many injured people with severe neurological disabilities. There are no satisfactory neuroprotective treatments. The present experiments demonstrated that administration of the C16 peptide (KAFDITYVRLKF (SEQ ID NO:2)) is neuroprotective after nervous system injury (e.g., traumatic injury). Thus, certain embodiments of the present invention are directed to methods of using the C16 peptide in neuroprotective treatments. Certain embodiments of the invention relate to methods of using variants of the C16 peptide. Certain embodiments of the invention relate to methods of using a peptide that comprises the YVRL (SEQ ID NO:1) sequence. The compounds of the present invention can also be used to treat neurodegenerative disease and inflammatory conditions.

Spinal cord injury results in loss of function and progressive secondary tissue degeneration, leaving many injured people with severe neurological disabilities. There are no satisfactory neuroprotective treatments. Disclosed herein are the neuroprotective effects of an angiopoietin-1 (Ang-1) mimetic and the improved neuroprotective effects demonstrated following co-administration of C16.

The only current neuroprotective treatment for spinal cord injury (SCI) is 24-48 hours of intravenous treatment with high doses of methylprednisolone, which has severe side-effects due to its general immune-suppressive actions. Further, methylprednisolone is not FDA approved for SCI and increasingly the efficacy of methylprednisolone is being questioned. Ang-1 and C16 are thought to act through a very selective receptor targets on the endothelial cells, thus limiting the potential side effects. Further, reagents that reduce vascular leakiness have not been tested, making Ang-1 unique. Minocycline reportedly has some neuroprotective effects for myelin but this effect is only temporary and certain axon pathways are not protected. Protection of axons is important for human voluntary function. Thus, current FDA-approved neuroprotective treatments for most degenerative disorders are non-existent.

Inflammation is thought to play a detrimental role in a large variety of degenerative neurological diseases, such as Alzheimer's disease and multiple sclerosis. Ang-1 and C16 should be useful to treat multiple sclerosis as this disease is characterized by bouts of disease linked to inflammation. Multiple sclerosis is treated with β-interferon, which is a protein with low bioavailability in the CNS, perhaps explaining its relatively low effectiveness. Stroke and traumatic brain injury (TBI) are also potential conditions that should be amenable to treatment with Ang-1 and C16 as those conditions are characterized by edema and detrimental post-injury inflammation.

Because Ang-1 and C16 can be administered to a location outside of the central nervous system, e.g., intravascularly, Ang-1 and C16 may have better bioavailability at the intended target the vasculature than many other peptide or protein based treatments that attempt to target cells on the other side of the blood-brain-barrier.

Ang-1 may reduce endothelial cell death and dysfunction thereby reducing ischemia, edema, and inflammation. It is thought that C16 targets the last step in transmigration of leukocytes, thus reducing infiltration through a very direct mechanism. This differential activity might explain the increased effectiveness of the combination of Ang-1 and C16 is improved. The treatments also might be more direct and more effective in reducing inflammatory signals, as compared to NSAIDS.

Anti-inflammatories are thought to be useful in a large array of diseases with some having no satisfactory treatments, e.g., rheumatoid arthritis.

Inhibition of leukocyte transmigration may reduce wound healing and general immune surveillance. Limiting the treatment period to a shorter duration may be useful in the case of acute disorders such as spinal cord injury, and possibly MS, stroke, trauma, and cancer.

Thus, certain embodiments of the present invention provide the use of Ang-1 and C16 to protect endothelial cells, thereby protecting the spinal cord after injury.

Administration of the C16 peptide (KAFDITYVRLKF (SEQ ID NO:2)) is neuroprotective after nervous system injury (e.g., traumatic injury). C16 is a 12 amino acid peptide homologous to a portion of the mouse and human laminin gamma 1 chain and has avb3 and α5β1 intregrin agonist activity.

Thus, certain embodiments of the present invention are directed to methods of using Ang-1 and the C16 peptide in neuroprotective treatments. Certain embodiments of the invention relate to methods of using variants of Ang-1 or the C16 peptide. Certain embodiments of the invention relate to methods of using a peptide that comprises the YVRL (SEQ ID NO:1) sequence. The compounds of the present invention can also be used to treat neurodegenerative disease and inflammatory conditions.

Certain embodiments of the present invention relate to treatments utilizing angiopoietin-1 (Ang-1), or a functional analog of Ang-1. A functional analog of Ang-1 is a molecule that possesses similar biological activity to Ang-1, e.g., by binding to and activating Tie2. An example of a functional analog of Ang-1 is recombinant human ANG1^{4FD} or Ang-1 TFD (also named Human BowAng1 Fc). It contains two human Ang1 fibrinogen-like domains fused to a human Fc domain to produce a multimer, which more efficiently activates Tie2 than a monomeric Ang1 domain. In some embodiments, a functional analog of Ang-1 includes in its structure about the 215 amino acids of the F1-domain or the fibrinogen-like domain of Ang-1. In some embodiments, a functional analog of Ang-1 includes in its structure about the 215 amino acids of the coiled-coil domain of Ang-1. In some embodiments, a functional analog of Ang-1 includes in its structure about the 50 amino acids of the N-terminal domain of Ang-1. Please also refer to Davis et al, Nature Structural Biology, 10(1), 38-44 (2003)) and U.S. Pat. No. 6,455,035 for discussions of Ang-1. In some embodiments, a functional Ang-1 analog would consist of shorter peptides or chemicals that specifically bind to Tie2 resulting in activation of the latter. Such shorter peptides could include the active sites in the Ang1 domains. Other peptides or certain chemicals could mimic the structure of the binding and activating sites of Ang-1.

Ang-1, C16, their derivative peptides and functional analogs, and compositions mimicking the active site or combinations of Ang-1 and C16 reagents might be neuroprotective agents for: traumatic injuries including but not limited to spinal cord injury and head trauma, as well as for ischemic stroke; neurodegenerative disorders, including but not limited to, Parkinson's disease, Huntington's disease, Multiple sclerosis, retinitis pigmetosa, uveitis, and peripheral neuropathies, among which are those associated with diabetes. Such compounds may also be useful in facilitating effects of treatments for regeneration or plasticity in a variety of neurological disorders. Such compounds may also be useful as diagnostic tools or vehicles to deliver other treatments. Such compounds may also be useful anti-inflammatory agents for any human or veterinarian disease. Such compounds may also be used as adjuvants anti-cancer drugs involving leukocyte derived neoplasia. For example, keeping cancerous leukocytes within the blood circulation might enable intravenous drugs to be more effective.

“Biological activity”, “bioactivity”, “activity”, and “biological function” are used interchangeably herein. In certain embodiments, biological activity means that a compound has activity at a specific receptor, e.g., is an agonist, at the αvβ3 and/or α5β1 integrin receptor. Biological activities can include binding to the receptor(s). αvβ3 or α5β1 integrin receptor bioactivity can be modulated (increased or decreased) by directly affecting the receptor.

The terms “protein,” “peptide” and “polypeptide” are used interchangeably herein.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also includes peptides with reduced peptide bonds, which can prevent proteolytic degradation of the peptide. Also, the term includes the amino acid analog α-amino-isobutyric acid. The term also includes natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

In certain embodiments, the peptides are modified by C-terminal amidation, head to tail cyclic peptides, or containing Cys residues for disulfide cyclization, siderophore modification, or N-terminal acetylation.

The term “peptide” describes a sequence of amino acids or peptidyl residues, e.g., 4 to 20 amino acids or peptidyl residues. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

By “variant” peptide is intended a peptide derived from the native peptide by deletion (so-called truncation) and/or addition of one or more amino acids to the N-terminal and/or C-terminal end of the native peptide; deletion and/or addition of one or more amino acids at one or more sites in the native peptide; and/or substitution of one or more amino acids at one or more sites in the native peptide. The peptides of the invention may be altered in various ways including, e.g., amino acid substitutions, deletions, truncations, and insertions. Methods for such manipulations are generally known in the art. For example, amino acid sequence variants of the peptides can be prepared by mutations in the DNA that encodes the amino acids. Methods for mutagenesis and nucleotide sequence alterations are well known in the art. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spatial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains.

Nucleic Acids of the Present Invention

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

The invention encompasses isolated and/or substantially purified nucleic acid or proteins, which may be included in compositions. In the context of the present invention, an “isolated” or “purified” DNA molecule or an “isolated” or “purified” protein is a DNA molecule or protein that exists apart from its native environment and is therefore not a product of nature. An isolated DNA molecule or protein may exist in a purified form or may exist in a non-native environment such as, for example, a transgenic host cell or bacteriophage. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

The term “chimeric” refers to any gene or DNA that contains 1) DNA sequences, including regulatory and coding sequences that are not found together in nature or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular cell to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes can comprise transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

“Chromosomally-integrated” refers to the integration of a foreign gene or DNA construct into the host DNA by covalent bonds. Where genes are not “chromosomally integrated”, they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or amino acids sequences: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. For example, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a known mathematical algorithm. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (available on the world wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world-wide-web at ncbi.nlm.nih.gov. Alignment may also be performed manually by visual inspection.

For purposes of the present invention, comparison of sequences for determination of percent sequence identity to the sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, at least 90%, or at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl: Tₘ81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

Therapeutic Compositions

The neuroprotective compound(s) (e.g., Ang-1, functional analogs of Ang-1, C16, variants of C16, peptides that comprise the YVRL (SEQ ID NO:1) sequence, or other agents that activate the αvβ3 and/or α5β1 integrins) can be formulated as pharmaceutical compositions and administered to a mammalian host, such as a human patient, in a variety of forms adapted to the chosen route of of administration. The neuroprotective compound(s) could also be administered to other types of mammals in need thereof, such as dogs, cats, horses, donkeys, mules, cows, sheep, goat, camel, etc.

In certain embodiments, the neuroprotective compound(s) can be administered in combination with an anti-inflammatory adjuvant, a blood vessel protectant, and/or another neuroprotective agent. In certain embodiments, the neuroprotective compound(s) can be administered in combination with methylprednisolone. The neuroprotective compound(s) may be administered, e.g., intrathecally, intraocularly, intravenously or intraperitoneally by infusion or injection. Solutions of the active compound or its salts can be prepared in phosphate buffered saline or saline, optionally mixed with a nontoxic surfactant. Ang-1 can be dissolved in physiological buffers and C16 can be dissolved in 0.3% acetic acid, and physiological pH can be re-establish by adding NaOH and then adding 1:1 PBS. Dispersions can also be prepared in glycerol, liquid polyethylene glycols, triacetin, and mixtures thereof and in oils. Under ordinary conditions of storage and use, these preparations contain a preservative to prevent the growth of microorganisms. Some of the compounds used in the present invention are not stable after dissolving them, so a dry formulation would need to be activated by bringing it into solution before administration.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the therapeutic compound(s) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

The desired dose may conveniently be presented in a continuous or single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention also provides a kit comprising Ang-1, or a functional analog thereof, and C16, or a pharmaceutically acceptable salt thereof, packaging material, and optionally instructions for administering Ang-1, or a functional analog thereof and C16 or the pharmaceutically acceptable salt thereof to an animal to treat neural injury (e.g., traumatic neural injury) or a degenerative disorder.

The neuroprotective compounds (e.g., C16, variants of C16, or peptides that comprise YVRL (SEQ ID NO:1)) can be formulated as pharmaceutical compositions and administered to a mammalian host, such as a human patient, in a variety of forms adapted to the chosen route of administration. The neuroprotective compound could also be administered to other types of mammals in need thereof, such as dogs, cats, horses, donkeys, mules, cows, sheep, goat, camel, etc.

The invention also provides a kit comprising C16, or a pharmaceutically acceptable salt thereof, at least one other therapeutic agent, packaging material, and instructions for administering C16 or the pharmaceutically acceptable salt thereof and the other therapeutic agent or agents to an animal to treat neural injury (e.g., traumatic neural injury) or a degenerative disorder.

Concentrations and Duration of Treatment with Compound(s)

The concentration of the neuroprotective compound(s) will vary depending on the condition to be treated and/or the mode of administration. Based on intravenous dosing in 30 gram mice, about 3 milligram per day per kilogram may be used. In certain embodiments, 0.1-30 mg/kg/day can be used for i.v. administration. The concentration used for topical applications may the same or may be higher.

In certain embodiments, the compound is administered for a period of less than six weeks. In certain embodiments, the compound is administered for a period of about one to four weeks. In other embodiments, such as to treat a degenerative disease such as multiple sclerosis, the compound will be administered for an extended period of time, such as for several years, or for the life of the patient.

Spinal Cord Injury or Brain Injury

For example, if the neuroprotective compound(s) is administered to treat a spinal cord injury or brain injury (e.g., traumatic injury), then the neuroprotective compound(s) can be administered at a dosage of less than about 10 mg/kg/day. In certain embodiments, the neuroprotective compound(s) can be administered at a dosage of 1 ng/kg/day to 30 mg/kg/day, or any integer value in between. For example the dosage may be at a range of about 1 to 3 mg/kg/day, or of about 3 mg/kg/day to 10 mg/kg/day. In certain embodiments, the neuroprotective compound(s) can be administered at a dosage of 3 mg/kg/day mouse dose. In such situations, the neuroprotective compound(s) can be administered intrathecally or intravenously. In certain embodiments, the neuroprotective compound(s) is administered within about 0-48 hours of injury. In certain embodiments, the neuroprotective compound(s) is administered within about 2-24 hours of injury. In certain embodiments, the neuroprotective compound(s) is administered within about 3-12 hours of injury. In certain embodiments, the neuroprotective compound(s) is administered within about 3-5 hours of injury.

As used herein, the term “traumatic injury” is defined as encompassing a non-severing or partially severing injury to the tissue, e.g., spinal cord or brain tissue or peripheral nerves. Examples of such injuries include a contusive injury, bruise, severe inflammation or other type of injury that does not cut the tissue. Other examples include a partial laceration, which can lead to secondary neural damage.

Multiple Sclerosis

The neuroprotective compound(s) may be administered to treat multiple sclerosis. To treat multiple sclerosis, the neuroprotective compound(s) can be administered at a dosage of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. In such situations, the neuroprotective compound(s) can be administered intrathecally or intravenously. When treating multiple sclerosis, the neuroprotective compound(s) may be administered for an extended period of time, such as for a period of days, weeks or years, or for the lifetime of the patient. The neuroprotective compound(s) may be administered as a series of injections or as an intravenous infusion during disease exacerbations.

Peripheral Nerve Injury

The neuroprotective compound(s) may be administered to treat a peripheral nerve injury. If the neuroprotective compound(s) is administered to treat a peripheral nerve injury, then the neuroprotective compound(s) can be administered at a dosage of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. In such situations, the neuroprotective compound(s) can be administered intrathecally, intravenously, or injected directly into the site of injury. In certain embodiments, the neuroprotective compound(s) is administered within about 0-48 hours of injury. In certain embodiments, the neuroprotective compound(s) is administered within about 2-24 hours of injury. In certain embodiments, the neuroprotective compound(s) is administered within about 3-12 hours of injury. In certain embodiments, the neuroprotective compound(s) is administered within about 3-5 hours of injury.

Eye Injury Affecting the Optic Nerve Fibers

The neuroprotective compound(s) may be administered to treat an eye injury affecting the optic nerve fibers. If the neuroprotective compound(s) is administered to treat an eye injury, then the neuroprotective compound(s) can be administered at a dosage of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. In such situations, the neuroprotective compound(s) can be administered intrathecally, intravenously, or injected directly into the site of injury. In certain embodiments, the neuroprotective compound(s) is administered within about 0-48 hours of injury. In certain embodiments, the neuroprotective compound(s) is administered within about 2-24 hours of injury. In certain embodiments, the neuroprotective compound(s) is administered within about 3-12 hours of injury. In certain embodiments, the neuroprotective compound(s) is administered within about 3-5 hours of injury.

Ischemic or Hemorrhagic Stroke

The neuroprotective compound(s) may be administered to treat ischemic or hemorrhagic stroke. If the neuroprotective compound(s) is administered to treat ischemic or hemorrhagic stroke, then the neuroprotective compound(s) can be administered at a dosage of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. In such situations, the neuroprotective compound(s) can be administered intrathecally, intravenously, or injected directly into the site of injury. In certain embodiments, the neuroprotective compound(s) is administered within about 0-48 hours of injury. In certain embodiments, the neuroprotective compound(s) is administered within about 2-24 hours of injury. In certain embodiments, the neuroprotective compound(s) is administered within about 3-12 hours of injury. In certain embodiments, the neuroprotective compound(s) is administered within about 3-5 hours of injury.

Skin Burn Injury

The neuroprotective compound(s) may be administered to treat a skin burn. The neuroprotective compound(s) may be administered topically, intrathecally, intravenously, or injected directly into the site of injury. The concentration of the neuroprotective compound(s) may vary depending on the mode of administration. If the neuroprotective compound(s) is administered topically, then the neuroprotective compound(s) may be administered at a dosage of less than about 100 mg/kg/day, such as at a range of 1 ng/kg/day to 300 mg/kg/day or any integer value in between. For example the dosage may be at a range of about 10 to 30 mg/kg/day, or of about 30 mg/kg/day to 100 mg/kg/day. If the neuroprotective compound(s) is administered intrathecally, intravenously, or injected directly into the site of injury, then the dosage can be at a concentration of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. In certain embodiments, the neuroprotective compound(s) is administered within about 0-48 hours of injury. In certain embodiments, the neuroprotective compound(s) is administered within about 2-24 hours of injury. In certain embodiments, the neuroprotective compound(s) is administered within about 3-12 hours of injury. In certain embodiments, the neuroprotective compound(s) is administered within about 3-5 hours of injury.

Pre-Treatment Prior to Surgery

The neuroprotective compound(s) may be administered as a pre-treatment prior to surgery. The neuroprotective compound(s) may be administered topically, intrathecally, intravenously, or injected directly into the site of surgery. The concentration of the neuroprotective compound(s) may vary depending on the mode of administration. If the neuroprotective compound(s) is administered topically, then the neuroprotective compound(s) can be administered at a dosage of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. If the neuroprotective compound(s) is administered intrathecally, intravenously, or injected directly into the site of injury, then the dosage can be at a concentration of less than about 10 mg/kg/day, such as at a range of 1 ng/kg/day to 30 mg/kg/day or any integer value in between. For example the dosage may be at a range of about 1 to 3 mg/kg/day, or of about 3 mg/kg/day to 10 mg/kg/day. In certain embodiments, the neuroprotective compound(s) is administered within about 0-48 hours of surgery. In certain embodiments, the neuroprotective compound(s) is administered within about 2-24 hours of surgery. In certain embodiments, the neuroprotective compound(s) is administered within about 3-12 hours of surgery. In certain embodiments, the neuroprotective compound(s) is administered within about 3-5 hours of surgery.

Inflammatory Conditions

The neuroprotective compound(s) may be administered to treat an inflammatory condition, such as uveitis or certain inflammatory peripheral neuropathies including Guillain-Barre syndrome, as well as disorders where inflammation is thought to play a detrimental role such as Alzheimer's disease.

The invention will now be illustrated by the following non-limiting Examples.

### Example 1

### An αVβ3 Integrin Agonist Reduces Leukocyte Transmigration and Rescues Blood Vessels, Myelin and Function after Spinal Cord Contusion in Mice

Spinal cord injury by contusion, compression or laceration causes progressive tissue loss due to secondary degeneration due to blood vessel dysfunction and inflammation. Endothelial cells and blood vessels are lost at the injury epicenter during the first 3 days after injury in rats and mice. In addition, surviving but damaged blood vessels become leaky due to disruption of the blood-spinal-barrier and endothelial dysfunction, contributing to detrimental edema and inflammation. Transmigration of leukocytes across the endothelial cell layer into the injured spinal cord tissue and subsequent microglia and macrophage activation contributes to local loss of myelin and tissue. Conversely, reduction of this inflammation improves tissue sparing and functional outcomes. The initial blood vessel loss is followed by an angiogenic response at the epicenter which is maintained up to 14 days in mice but regresses from day 7-14 from day 7-14 in rats. It was unknown whether protection of the damaged blood vessels during the first few days or stimulation of angiogenesis would lead to improved tissue sparing and function.

Integrins are heterodimer transmembrane receptors which have a reciprocal functional interaction with growth factor receptors. The αvβ3 integrin (vitronectin receptor) is present on the luminal surface of endothelial cells and is important for angiogenesis. Endothelial cell survival is promoted by αvβ3 integrin during angiogenesis. The interaction between extracellular matrix molecules, such as laminin, and integrin receptors is important for attachment and survival of various cells, including endothelia. Endothelial attachment is disrupted after spinal cord injury. A peptide named C16, representing one of the functional domains of the γ1 laminin chain, selectively activates αvβ3 and α5β1 integrin to promote angiogenesis in vitro and in the chick chorioallantoid membrane.

The αvβ3 integrin potentially also plays a role in inflammation as it contributes to leukocyte transmigration across endothelia in response to some but not all inflammatory activators. On the other hand, αvβ3 integrin occupancy reduces monocyte binding to ICAM, which is important for transmigration. Some leukocytes, including monocytes, also can express αvβ3 integrin. Therefore, the effects of αvβ3 stimulation on inflammation are not predictable.

Here, the C16 αvβ3 agonist peptide was tested for its ability to preserve perfused blood vessels and reduce secondary degeneration and functional deficits after a contusive spinal cord injury in adult mice.

Materials and Methods

Animals and Experimental Design

Female C57BL/6 mice were purchased from Jackson Laboratory (Bar Harbor, Me.) and were 7-11 weeks old weighing 16-24 g at the time of spinal cord injury. Age and weight were the same between groups within an experiment. All animal procedures were performed according to University of Louisville IACUC protocols and the National Institute of Health guidelines. All invasive procedures were performed under deep anesthesia obtained by an intraperitoneal injection of Avertin (0.4 mg 2,2,2-tribromoethanol in 0.02 ml of 1.25% 2-methyl-2-butanol in saline per gram body weight, Sigma-Aldrich, St. Louis, Mo.).

A total of 133 mice were used. Surgeries, behavioral measurements and quantification of histological results were done by investigators blind to the treatment. Treatment solutions were assigned in a randomized order and were prepared and coded by someone different than the surgeon.

To determine whether and when αvβ3 and α5β1 intergins were expressed in blood vessels early during the treatment, mice received a T9 contusion and horizontal sections through the injured spinal cord were analyzed after 1 or 3 days (n=4 each). These mice received in an intravenous injection of IB4 lectin 30 minutes before euthanasia.

To determine whether C16 had neuroprotective effects, mice received a contusion at vertebral level T9 and received daily intravenous injections with vehicle (n=10) or C16 at 100 μg/day (n=11), starting immediately after the injury.

After 7 days, horizontal sections through the spinal cord were analyzed for a reduction in the volume of tissue loss. To more readily assess myelin loss and inflammatory responses at the epicenter transverse sections were used for all subsequent experiments.

To test whether 100 μg/day C16 had neuroprotective and functional effects at 7 days after injury, other groups of mice received vehicle (n=13), C16 (n=8) or an inactive SP3 peptide (100 μg/day; n=7). These mice were tested for overground locomotion using the Basso Mouse Scale (BMS) before the contusion and on the day of euthanasia.

To label endothelial cells of perfused blood vessels, 4 of the vehicle and 4 of the C16 mice received an intravenous injection of the lectin LEA 20 minutes before euthanasia. Next, the investigators determined the most effective dose of C16, by injecting vehicle (n=5), 30 μg/day C16 (n=5), 100 μg/day C16 (n=9), or 300 μg/day C16 (n=5) daily and analyzing the cords 7 days after injury.

To determine whether a single injection of C16 would be neuroprotective, vehicle (n=8) or 100 μg/day C16 (n=9) was injected over 7 days. These mice were tested using the BMS before the contusion and on the last day.

last day.

To determine whether C16 had lasting neuroprotective effects, mice received daily injections with vehicle (n=8) or 100 μg/day C16 (n=9) for 14 days. The treatment was started 4 hours after the spinal cord injury consistent with the time it takes to diagnose most injured humans in the developed world. These mice were evaluated every week by BMS and were euthanized after 6 weeks. LEA was injected 20 minutes before euthanasia.

To determine whether C16 could protect blood vessels, contused mice received an injection of vehicle (n=5) or 100 μg/day C16 (n=5) immediately after the injury and received an intravenous injection of LEA after 24 hours, 20 minutes before euthanasia. Normal (n=3) and 7 day sham operated mice (n=5) also received intravenous LEA injections.

Spinal Cord Injury

The mice were anaesthetized and their backs shaved and cleansed with a betadine (Purdue Products L.P., Stamford, Conn.). Lacrilube ophthalmic ointment (Allergen, Irvine, Calif.) was placed on their eyes to prevent drying and 50 mg/kg of gentamicin (Boehringer Ingelheim, St. Joseph, Mo.) was administered i.m. to reduce infections. After a midline incision and laminectomy of the T9 vertebra, spinal cord contusions were induced after using the Infinite Horizon (IH) device with the impact force set at 50 kdyn (PSI, Lexington, Ky.). The vertebral column was stabilized in a frame with rigid steel clamps inserted under the transverse processes. After the injury, the muscles were closed in layers, the skin incision was closed with 7 mm metal wound clips and 2 ml lactated Ringer's solution were given subcutaneously. Bacitracin zinc antibiotic ointment (Altana, Melville, N.Y.) was applied to the incision area. Food available was placed on the bottom of the cage, and water bottles with long sipping tubes were used. Buprenorphine (0.05 mg/kg) was given subcutaneously at 48 hours post-injury to reduce pain. Bladders were manually expressed twice daily until the mice had regained partial voluntary or autonomic voiding, at which time they were reduced to once a day manual expression until full voluntary or autonomic voiding was obtained. Surgeries were performed at room temperature with the mice positioned on a heating pad to maintain body temperature. After the surgery, surgery, mice were placed on fresh alpha dry bedding with cages placed on a water circulating thermal pad (37° C.) overnight before being returned to the animal care facility. Metal sutures were removed after 14 days.

Intravenous Injections

For intravenous injection on the day of spinal cord injury, a midline incision was made in the ventral neck area and one of the jugular veins was exposed by blunt dissection. After ligation of the jugular vein, 100 μl of sterile vehicle or vehicle containing sterile C16 peptide (KAFDITYVRLKF (SEQ ID NO:2); 10-300 μg; synthesized by Peptides International, Louisville, Ky.), or SP3 peptide (RFSVAVSSHYPFWSR (SEQ ID NO:3); 100 μg; synthesized by Sigma-Genosys, St. Louis, Mo.) was administered.

C16 is very selective as it was shown by affinity chromatography and immunoprecipitation to bind only to αvβ3 and α5β1 integrins, and not α1, α2, α3, α6, β4, αvβ5. Moreover, C16 activity is blocked by αvβ3 and α5β1 antibodies in vitro. The investigators did not use C16 based control peptides, as both scrambled C16 and reverse C16 have biological activity. Instead, the investigators used SP3 peptide which is an inactive scrambled form of an α6β1 binding peptide and has no known activity after spinal cord injury in rats or on monocyte transmigration in vitro (see below).

To improve solubility of the peptides, they were dissolved in distilled water with 0.3% acetic acid. Afterwards, the peptide was sterilized through a 0.22 μm disc filter and the solution neutralized by adding NaOH. This solution was buffered by adding an equal volume of sterile PBS. The vehicle was prepared in the same manner without adding the peptide. After the jugular vein injection, the skin was closed by metal sutures. On the following 6 or 13 days the solutions were injected via the tail vein injection. Ready access to the tail veins was achieved by starting injections at the caudal end of the base of the tail and into one vein. Injection sites were moved to the alternate left or right sides and increasingly more rostral on subsequent days.

To pre-label perfused blood vessels, 100 μg in 100 μl of FITC-conjugated LEA (Lectin from Lycopersicon esculentum; Sigma, St. Louis, Mo.) was injected into the other jugular vein 30 minutes before euthanasia. In the mice of the time mice of the time course experiment, 100 μg in 100 μl of FITC-conjugated IB4 lectin (Sigma) was injected.

To determine potential effects of C16 on the number of peripheral leukocytes, 1 ml blood was drawn from the heart just before perfusion-fixation in mice that had been treated with vehicle (n=3) or C16 (n=2) for 7 days treated. The blood was collected in EDTA-coated tubes and blood counts were performed by Drew Scientific Inc. (Oxford, Conn.).

Functional Testing

Functional recovery from the spinal cord contusion was determined weekly by open-field overground locomotor performance using the Basso Mouse Scale (BMS; Basso et al., 2006, J Neurotrauma 23:635-659), with scores ranging from 0 (complete paralysis) to 9 (normal mobility). Left and right sides are scored separately and averaged for each mouse. Weight bearing is seen at a score of 5 but not at 4. Locomotor function was measured by placing the mouse for 4 min in the center of a circular enclosure (90 cm in diameter, 7 cm wall height) made of molded plastic with a smooth, non-slip floor. Before each evaluation, the mice were examined carefully for perineal infections, wounds in the limbs, or tail and foot autophagia, as they could influence stepping. Such mice were excluded from the BMS test. The mice were tested for baseline values before surgery. The mice in the 7-day experiments were tested on the day of euthanasia and the mice in the 6 week chronic group were tested every week starting on day 8 or 9 after the contusion.

Histological Procedures

Mice were perfused transcardially with 10 ml PBS followed by 20 ml of 4% paraformaldehyde in 0.1M phosphate buffer (PH 7.4). Afterwards, the spinal cords were carefully dissected out and 1 cm segments containing the injury site were post-fixed for 4 hours and then cryoprotected in 30% phosphate buffered sucrose overnight. Up to 10 segments were embedded in TissueTek® (Sakura Finetek, Torrance, Calif.) with their injury sites aligned. This ensures that groups within an experiment are processed for histology in the same manner. Twenty consecutive 20 μm transverse sections per 1 mm rostro-caudal distance along the spinal cord axis were cut on a cryostat and thaw-mounted onto charged microscope slides. In one set of mice the cord was cut in the horizontal plane. The sections were stored in sequence at −20° C. until further use.

To detect myelin in white matter tracts, one of every five of the transverse sections at each rostro-caudal 1 mm level were stained with a modified eriochrome cyanine (EC) staining protocol (Rabchevsky et al., 2001, J Neurotrauma 18:513-522). After thawing and drying for 1 to 2 hours in a slide warmer at 37° C., the slides were placed in xylene at room temperature for 5 minutes, then through graded ethanol solutions (twice each in 100 and 95% ethanol, once in 70% ethanol, and twice in ddH₂O) and stained with EC solution (0.2% eriochrome cyanine RS, 0.5% sulfuric acid, and 0.4% ferric ammonium sulfate) for 30 minutes. Afterwards, the slides were gently washed in running tap water for 5 minutes, and then briefly rinsed in ddH₂O. The slide was differentiated in 5% ferric ammonium sulfate for 5 to 10 minutes, briefly rinsed in ddH₂O, dehydrated briefly through graded ethanol solutions, cleared through xylene, and the sections coverslipped in Entellan® embedding agent (Electron Microscopy Sciences, Hatfield, Pa.). The injury epicenter was determined for each mouse by the rostro-caudal level that contained the least amount of spared myelin per transverse section. This epicenter level was used to align the other histological measurements for each mouse.

Adjacent sections at each mm level were processed for double- or triple immunofluorescent staining to detect CD45 (leukocytes), CD68 (activated microglia/macrophages), PECAM1 (endothelial cells), αv, β3 or α5β1 integrin, or in some cases laminin (to define the so-called heterodomain or area of tissue loss). Slides were warmed for 20 minutes on a slide warmer, a ring of wax applied around the sections with a PAP pen (Invitrogen™, Carlsbad, Calif.) and the slides rinsed in 0.1 M Tris-buffered saline (TBS) for 10 min. After blocking non-specific staining with 10% donkey serum in TBS containing 0.3% Triton X-100 (TBST) for 1 hour at room temperature, sections were incubated overnight at 4° C. in TBST containing 5% donkey serum containing rat anti-CD45 (1:500, Cat# CBL1326, Chemicon International, Temecula, Calif.), rat anti-CD68 (1:1000, Cat# MCA 1957, ABD Serotec, Kindlington, Oxford, UK) or rat anti-PECAM1 (1:500, Cat# 550274, BD Pharmingen, San Jose, Calif.), rabbit anti-αv integrin (1:1000, Cat# AB1930; Chemicon), rabbit anti-β3 integrin (1:100, Cat# AB1932, Chemicon), rat anti-α5β1 integrin (1:100, Cat# MAB1984, Chemicon), or rabbit anti-laminin IgG (1:300, Cat# L9393, Sigma). As a control, purified rat or rabbit IgG (IR-RB-IGG, IR-IB IGG, Innovative Research, NoVi, Mich.) was used instead of the primary antibody. Next, the sections were incubated in TBST containing 5% donkey serum and 1:500 of appropriate secondary antibodies (donkey TRITC-conjugated Fab fragments, Invitrogen™, Carlsbad, Calif.; Alexa594) for 1 h at room temperature. Finally, the sections were coverslipped with antifade Gel/Mount aqueous mounting media (SouthernBiotech, Birmingham, Ala.). In between steps, sections were washed 3 times for 10 minutes in TBS. To measure the volume of tissue loss as determined by laminin staining in the horizontal sections, the sections were processed using an ABC-DAB staining protocol.

Quantitative Measurements and Statistical Analyses

Sections were examined using a Leica DMIRE2 brightfield and fluorescence microscope and images digitized with an attached Spot RTKE camera (Diagnostics Inc., Sterling Height, Mich.). EC, CD45, CD68 and PECAM staining through the entire plane of the transverse sections was digitized using a 5× objective and the area occupied by the staining calculated for the three sections per rostro-caudal level by using the threshold feature of Scion Image software (Scion Corporation, Frederick, Md.). For analysis of LEA labeled (perfused) blood vessels, images of the dorsal column and adjacent gray matter and of the ventrolateral funiculus and adjacent gray matter were taken at the injury epicenter and at 1 mm rostral and caudal to it using a 20× objective. The area of LEA was determined with Scion Image. To provide a measure of the number of blood vessels, the number of LEA-positive vessels intersecting 100 μm spaced horizontal (5) and vertical (6) lines were counted in each image. To determine the volume of the heterodomain, every 5^{th} horizontal section was stained for laminin using DAB as substrate, the heterodomain was circled in each section as well the outline of the entire 10 mm length of the spinal cord segment using Neurolucida software (MBF Bioscience, Williston, Vt.). The software calculated the volume based on the section interval and the area per section and this was expressed as a percentage of the total 10 mm segment.

Statistical significant differences between groups were determined by t-Test or ANOVA with post hoc t-tests, and both it and regression analyses were performed using Excel (Microsoft Office XP Professional) or Sigmastat (Systat Software Inc., San Jose, Calif.) software. A p-value of less than 0.05 was considered statistically significant. Values for groups are presented as an average±standard error of the mean (SEM).

Transendothelial Migration Assay

Human aortic endothelial cells (Lonza, Wakersville, Md.) were plated on permeable filters in Transwell® culture plates (Costar, Cambridge, Mass.) at 4×10⁴ cells per well. They were grown for 72-96 hours to reach confluency at 37° C. in Dulbecco's modified Eagle's medium Ham's F-12 (DMEM F-12; BioWhittaker, Walkersville, Md.) plus EGM-2 growth factor supplements (EGM-2 SingleQuots™ from Lonza Walkersville Inc, containing hydrocortisone, hEGF, FBS, VEGF, hFGF-B, R3-IGF-1, ascorbic acid, heparin and gentamicin/amphotericin-B) and 10-20% fetal calf serum. Afterwards, the inner chamber of the Transwell® system was loaded with 2×10⁵ monocytic THP-1 cells (American Type Culture Collection, Manassas, Va.) and preincubated in medium with or without 15 ng/ml of TNF-α (PeproTech, Rocky Hill, N.J.) for 5 hr at 37° C. C16 peptide was added into the medium of the inner chamber at 25, 50, 100, 200, 400, 600 μM, and SP3 was added at 600 μM. Peptides were centrifuged to remove any precipitates before addition. The vehicle served as control. The monocytic THP-1 cells were allowed to transmigrate for 6 h at 37° C. and the number of THP-1 cells that crossed endothelial cell layer was counted. Values were derived from 3 wells per concentration and the experiment run in duplicate. To assess the functions of integrins, azide-free blocking antibodies against human αvβ3 (Cat# MAB1976Z, Chemicon; also known as LM609) and human α5 (Cat# MAB1956Z, Clone P1D6 Chemicon) integrin were added in a separate experiment without C16 peptide.

Results

Blood Vessels Express αvβ3 Integrin after Spinal Cord Contusion in Mice

To determine whether blood vessels in the injury epicenter could respond to C16 at day 1 and 3 post-injury, horizontal sections of mice injected with IB4 lectin were double-immunostained for PECAM1 (endothelial cells) and either αv or β3 integrin subunit or α5β1 integrin. There are no antibodies that recognize mouse αvβ3 heterodimers and the subunit antibodies could not be used for double-αv and β3 labeling as they were made in the same species. Twenty-four hours (and three days) after injury immunostaining for αv integrin subunit is seen at the epicenter on some blood vessels identified by intravenous injection of LEA lectin 30 minutes before histological processing. Many neurons also stain for αv integrin. These observations were confirmed by confocal microscopy. β3 integrin staining was more clearly detectable on blood vessels in and around the injury site. Staining for αv β3 integrin was also present in many neurons in the normal mouse but disappeared at the injury epicenter. Staining for α5β1 integrin was present in many neurons but not in LEA-positive blood vessels. The integrins did not seem to be present in infiltrating leukocytes, which are abundant at the epicenter three days following injury.

Intravenous C16 Injections Reduce the Volume of Tissue Loss after Spinal Cord Contusion

In mice, lost tissue is replaced by laminin-rich mesenchymal tissue forming a so-called heterodomain. A heterodomain is characterized by deposits of laminin by invading mesenchymal cells which replace lost spinal cord tissue in a mouse contused seven days before at T9. Compared to mice that had received daily intravenous injections of vehicle, the mice treated with C16 showed a less extensive heterodomain at seven days following injury. Blood vessels identified by laminin-positive basement membrane in the injury penumbra were preserved.

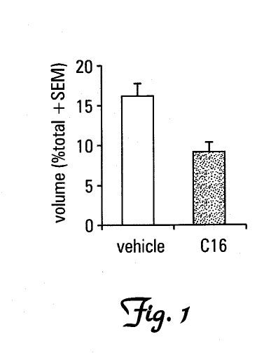

With vehicle treatment, tissue loss occurred over half the diameter of the spinal cord, whereas with C16 treatment damage appeared to be less in the outer regions of the spinal cord, including white matter tracts. Of note was the more normal architecture of the laminin-positive blood vessel plexus seen in the injury penumbra of C16 treated mice. The volume of the laminin-positive heterodomain showed a 43% reduction after C16 treatment compared to vehicle treatment (FIG. 1; p<0.005).

C16 Treatment Protects White Matter and Function 1 Week after Spinal Cord Injury

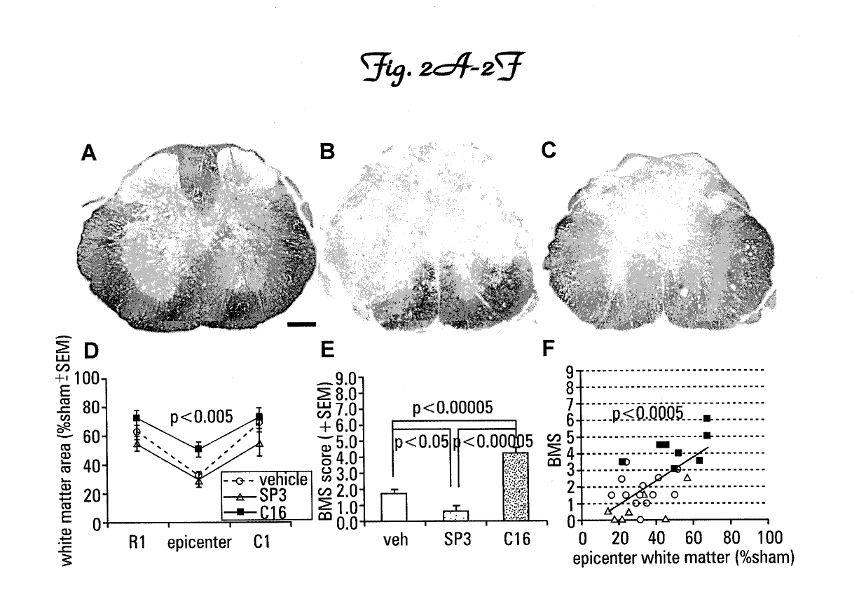

To more precisely analyze the white matter sparing, all the following experiments used transverse sections through the epicenter and at 1 mm distances from it. The epicenter was determined for each mouse by the rostro-caudal level along the spinal cord axis containing the minimum area of myelin as shown by EC staining. In mice treated with daily intravenous injections of vehicle (FIG. 2B) or control peptide SP3 for 7 days following the contusion, much of the myelin in the dorsal ⅔^{rd} of the spinal cord was lost. In some mice, the injury caused a “punch-out” injury where only the most ventrolateral white matter tracts remained intact. In contrast, in mice treated with C16, much of the ventral and lateral white matter was spared (FIG. 2C). The cross-sectional area of white matter at the injury epicenter was reduced to ˜33±3% (SEM) and 30±5% of sham operated mice in the vehicle and SP3 treated group of mice, respectively (FIG. 2D). With the C16 treatment, 52±5% of the epicenter white matter was spared, which was significantly different from the two control groups (p<0.005). The area of white matter was greater at 1 mm rostral, but not caudal, to the injury with C16 treatments (FIG. 2D). At 2 mm from the epicenter in both directions the myelin appeared normal in all groups. These mice were tested for locomotor function on the day of euthanasia, 7 days following injury. With vehicle treatment, the BMS score was 1.8±0.3, indicating a moderately to severe injury. The BMS score in the SP3 treatment group was lower (0.7±0.3; FIG. 2E; p<0.05) than in the vehicle treated group despite the same white matter sparing. This raises the possibility that SP3 has other, possibly systemic, effects that affect the BMS score. In sharp contrast, the C16 treated group had a BMS score of 4.3±0.3 which was significantly greater than that of the vehicle group (p<0.00001). Two out of the 8 C16-treated mice had a score of 5 or higher, indicating weight-bearing ability. A regression analysis using the data from all the mice in the three groups combined, showed a clear correlation between epicenter white matter sparing and BMS scores (FIG. 2F; p<0.0005).

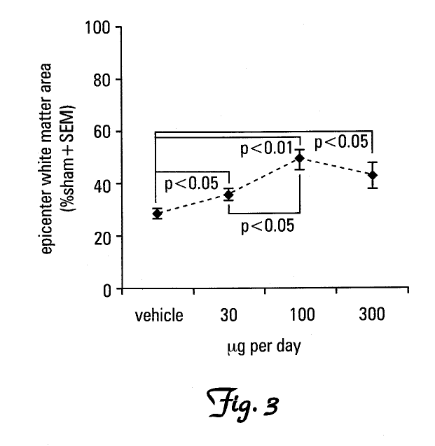

To determine the maximally effective dose of C16 for white matter sparing, mice were injected daily with vehicle, 30, 100 or 300 μg C16 over 7 days after the spinal cord contusion. These mice had 29±2, 36±2, 49±4 and 43±5% of white matter remaining at 7 days (FIG. 3). The values of all the C16 treated groups were significantly greater than that of the vehicle group (p<0.05, 0.001, 0.05). The 100 μg C16 group average was greater than that of the 30 μg group p<0.05) but not different from the 300 μg group. This suggests that 100 μg is the lowest dose with the maximum effect, and it was used in further experiments.

C16 Provides Lasting Functional Improvement and Neuroprotection after Spinal Cord Injury

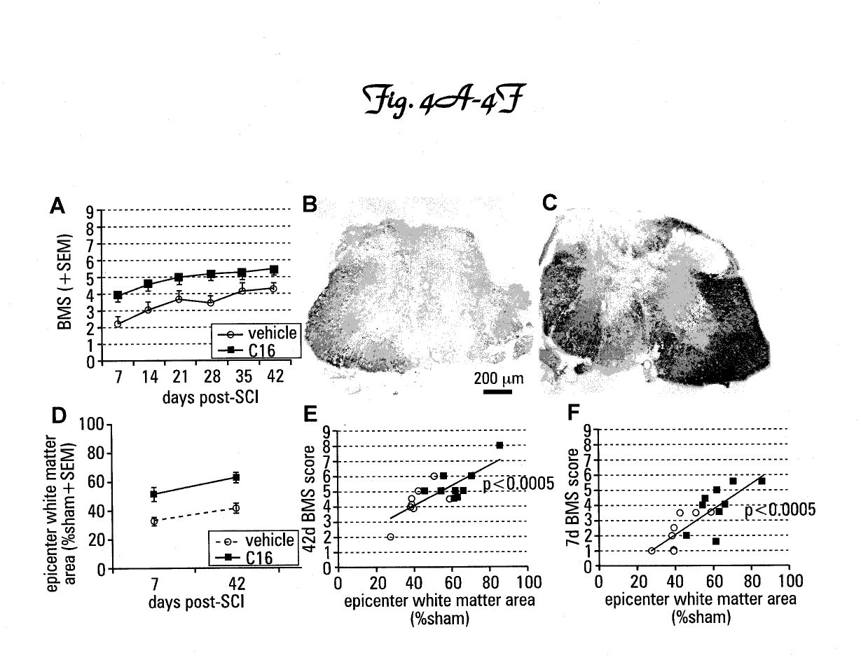

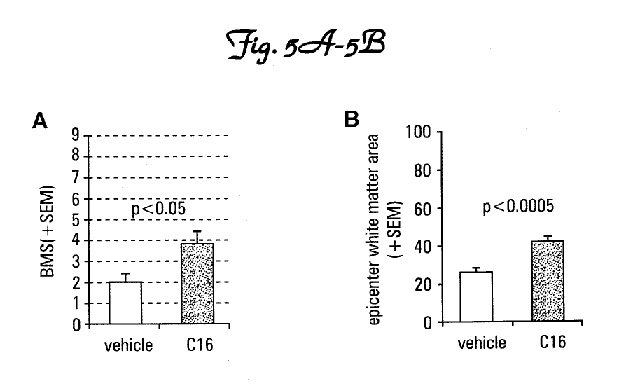

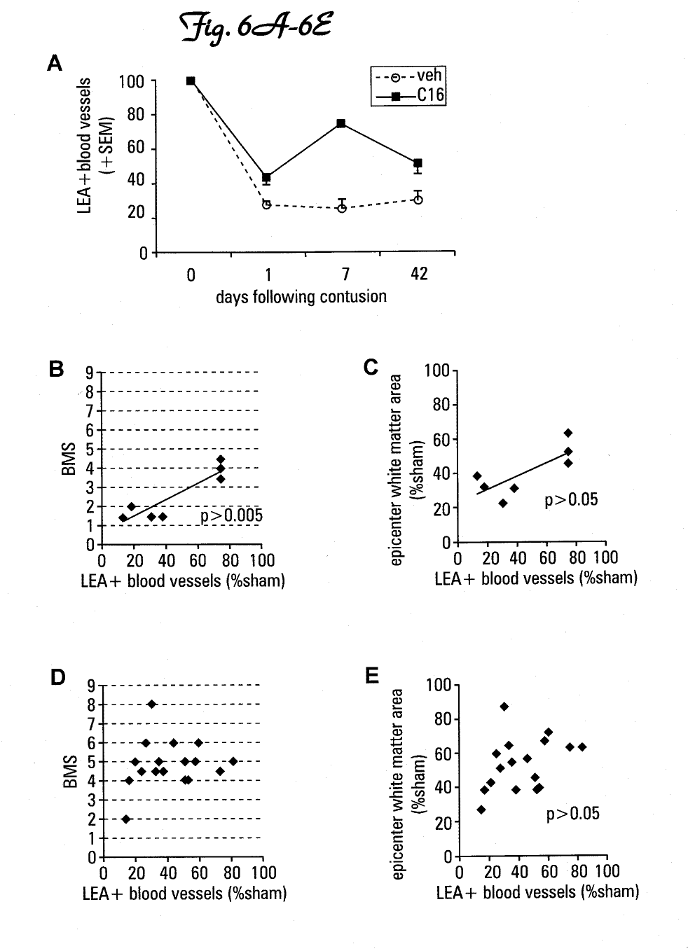

To determine whether C16 would provide lasting neuroprotection, mice received a contusion at T9 and daily intravascular injections with vehicle or 100 μg C16 over 14 days, starting 4 hours after the injury. They were then followed for another 4 weeks. At 7 days, C16 treated mice had a higher BMS score (3.9±0.5) than vehicle treated mice (2.3±0.4; p<0.01; FIG. 4A). Remarkably, 3 out of 9 C16 treated mice had a BMS score of 5 or above (weight-bearing) already one week after contusion compared to none of the 8 mice in the vehicle group. The improved locomotor function continued until week 6, when C16 treated mice on average were weight-bearing (BMS: 5.4±0.4) and vehicle treated mice not (4.3±0.4; p<0.05). Seven out of nine C16 treated mice had a BMS score of 5 or above (weight-bearing) compared to only 2 out of 8 in the vehicle group. The finding that the functional difference is seen as early as 7 days suggests that mechanisms during the acute and early sub-acute phase are affected by C16.

Histological analysis showed that the extent of spared white matter at the injury epicenter was greater in the C16 treated mice (FIG. 4B; 63±4% of sham) than in vehicle treated mice (FIG. 4C; 41±3%; p<0.0005). The extent of white matter sparing in the vehicle or C16 treated 7 day group (data from FIGS. 2A-2F) and these vehicle or C16-treated chronic mice, respectively, was not significantly different (FIG. 4D; p=0.07 and 0.09). If anything, the area of spared white matter was greater at the 42 day point. A regression analysis revealed a high degree of correlation between the area of spared white matter at 42 days and both the last (week 6; FIG. 4E; p<0.0005) and the first BMS performed 7 days following injury (FIG. 4F; p<0.0005). The average BMS scores did not differ at the 7 day point between the acute (7 day treated and euthanized) and chronic (6 week) group, confirming that they had received a similar injury. Together, these results suggest that C16 reduces degenerative mechanisms in the white matter and the resulting locomotor deficits predominantly over the first 7 days, during the early post-injury phase.

A Single C16 Injection is as Neuroprotective Suggesting Effects During the Acute Post-Injury Phase

To determine whether a single injection of C16 would be neuroprotective, mice received an intravenous injection of vehicle or C16 immediately after the T9 contusion and were analyzed after 7 days. The BMS score was higher in the C16 treated group (3.8±0.6) compared to the ones injected with a single bolus of vehicle (2.0±0.4; p<0.05; FIG. 5A) and not significantly different from C16 group treated for 7 days (4.3±0.3; p=0.26; data from FIG. 2E). Similarly, the area of spared white matter at the epicenter was greater in the single bolus C16 treated group (42±3%) compared to the ones injected with a single bolus of vehicle (26±2; p<0.0005; FIG. 5B) and not significantly different from C16 group treated for 7 days (51±5; p=0.06; data from FIG. 2D). Injected peptides are expected to be metabolized and/or excreted over the first day after injection. This suggests that C16 affects very early degenerative events such as blood vessel loss. In addition, the trend (p=0.06) of a difference between the white matter sparing seen after the single injection and the 7 day injections, left open a possibility that C16 also affects later degenerative mechanisms such as macrophage extravasation and activation.

C16 Treatment Results in More Functioning Blood Vessels Around the Injury Site

To determine whether C16 would affect the vasculature, PECAM stained sections at the epicenter were analyzed at 7 days after the contusion. Sham operated mice showed a normal blood vessel plexus, which was clearly disturbed by the contusion in vehicle treated but less in C16 treated mice. Probably more important to tissue protection is the question of how many functioning blood vessels are present. Therefore, sections at the epicenter (as determined by the minimum EC staining) and at 1 mm rostral and caudal from it were analyzed for the number of blood vessels that had bound LEA injected intravenously 30 minutes before euthanasia. Sham operated mice showed a clear blood vessel plexus. Twenty-four hours after a spinal cord contusion at T9, those injected intravenously with vehicle immediately after the injury showed a loss of blood vessels. The investigators observed loss of blood vessels at 1 day post-injury before, using injected LEA as a marker. In contrast, C16 injected mice appeared to have more perfused blood vessels. The number of LEA-positive blood vessels in the dorsal and ventrolateral regions of the penumbra was 28±1% of sham in vehicle-treated mice compared to 44±4% in C16 treated mice (p<0.005; FIG. 6A). This suggests that the C16 treatment can rescue blood vessels. At 7 days and 6 weeks after the injury, C16 treated mice also appeared to have more LEA-positive blood vessels than vehicle-treated mice. At 7 days, the number of LEA-positive blood vessels in the penumbral regions was greater in C16 treated mice (75±1% of sham/normal mice) than in vehicle treated mice (25±6%; p<0.0005; FIG. 6A). Mice treated for 14 days and analyzed at 6 weeks following injury had more blood vessels after C16 treatment than vehicle treatment (51±6 vs 30±5; p<0.01). The number of LEA-labeled blood vessels was greater at 7 than at 1 day after injury in the C16 treated group (p<0.005), but not in the vehicle treated group, suggesting that C16 can stimulate angiogenesis. The number of vessels in the C16-treated chronic mice was not different (p=0.054) compared to the C16 treated mice analyzed at 7 days (FIG. 6A), suggesting that some or the new blood vessels that had grown between day 1 and 7 were maintained up to 6 weeks post-injury.

At 7 days after injury, the total number of LEA positive blood vessels correlated with the extent of total white matter sparing (p=0.042; FIG. 6B) and the BMS scores (p<0.005; FIG. 6C). This raises the possibility that the improved vascularity contributed to increased white matter sparing leading to improved functional outcome. However, at 6 weeks after injury, the number of LEA positive blood vessels did not correlate with the white matter sparing or the BMS scores (p=0.17, p=0.57; FIGS. 6D, 6E). In contrast, white matter sparing correlated well with the BMS scores (FIG. 4B). Thus, increased vascularity alone is not sufficient to explain the lasting improvements in white matter sparing and function obtained with C16 treatments.

C16 Reduces Inflammation Following Spinal Cord Contusion

Inflammation is an Important Contributor to White Matter Loss after Spinal cord injury. The investigators therefore tested whether C16 would reduce inflammation by analyzing CD45 as a marker for peripheral leukocytes, i.e., for extravasation into the injured cord, and CD68, which is a marker for activation of the resident microglia and the infiltrated macrophages. In sham operated mice, essentially no immunostaining for CD45 and very little for CD68 could be detected. Contused mice treated with vehicle and analyzed 24 hours later showed a modest increase in CD45 and CD68 staining. In C16 treated mice, the extent of inflammation seemed slightly reduced. At 7 and 42 days after injury, the inflammatory response was greatly increased in vehicle treated mice over most of the cross-section of the spinal cord at the injury epicenter, whereas it was markedly attenuated in C16 treated mice.

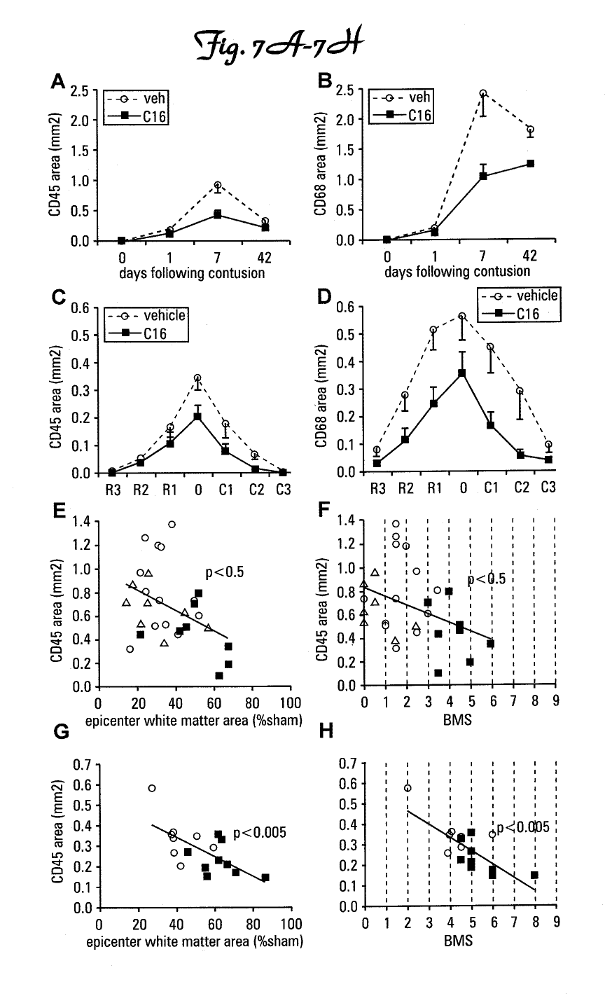

Quantification of the area of immunostaining at 1 mm distances from 3 mm rostral to 3 mm caudal from the epicenter showed that the contusion caused an increase in inflammation in vehicle treated mice with the largest extent seen at 7 days (FIG. 7A, 7B). In C16 treated mice the inflammatory response was reduced at all post-injury time points. At 24 hours the C16 treated mice had a 39% and 23% smaller area of CD45 and CD68 staining, respectively than vehicle treated mice (p<0.05, 0.005). At 7 days, values of C16 treated mice were 53% and 57% lower (p<0.005 each). Plots of the rostrocaudal distribution of the area of immunostaining seen at 7 days showed that C16 reduced the CD45 and CD68 area at the epicenter by 49% and 44% (p<0.05 each), respectively (FIG. 7C, 7D). The inflammation was also reduced at 1-2 mm away from the injury site. The control peptide SP3 had no significant effect on the inflammatory response seen at 7 days (p=0.27). Regression analyses showed that the extent of inflammation at 7 days post-injury correlated with the extent of white matter loss (FIG. 7E; p<0.05) and reduction in BMS scores (FIG. 7F; p<0.05). The single bolus injection of C16 had no significant effect on the CD45 area at 7 days after the injury but reduced the CD68 stained area by 26% compared to vehicle injections injections (p<0.01; not shown). The mice treated with C16 for 7 days clearly had a much reduced inflammatory response (a reduction of 53 and 57% for CD45 and CD68, respectively, compared to vehicle). This again suggests that C16 targets very early as well as later events during the 7 day post-injury period.

Chronic inflammation after spinal cord injury occurs in rodents and to a somewhat lesser extent in humans. Here, in vehicle treated mice the reduction in total area seen between day 7 and 42 was more extensive for CD45 than for CD68, but both markers were still found throughout large regions of the cord (FIGS. 7A, 7B). In the C16 treated group, the 7 and 42 day CD68 values were not significantly different, suggesting that microglial activation is suppressed less after termination of the treatment. Even so, the area of CD45 and CD68 immunostaining at 6 weeks was 32% and 30% less in C16 treated mice than in vehicle treated mice, respectively (p<0.05, p<0.0005; FIGS. 7A, 7B). The extent of chronic inflammation correlated with the reduced white matter sparing and the last BMS scores (FIGS. 7G, 7H), as it did in the 7 day post-injury mice (see above).

To determine whether the reduced inflammation might be due to a reduced number of peripheral leukocytes, blood was withdrawn from 2 mice treated with vehicle and 3 mice treated with C16 for 7 days. Blood counts showed that the white cell counts were within normal range for both the vehicle and C16 groups (Table 1). Some mice had low red blood cell and thrombocyte counts probably due to bleeding caused by spinal cord surgeries.

TABLE 1
C16 does not affect systemic leukocyte counts.

| | vehicle A | vehicle B | vehicle C | C16 A | C16 B |
|---|---|---|---|---|---|
| Leukocytes | | | | | |
| total | low | N | N | N | N |
| neutrophils | N | N | N | N | N |
| lymphocytes | N | N | N | N | N |
| monocytes | N | N | high | N | N |
| eosinophils | N | N | high | N | N |
| basophils | N | N | N | N | N |
| Erythrocytes | low | low | N | N | N |
| Thrombocytes | low | low | N | low | low |

Mice were injected daily with vehicle (n = 3) or C16 (n = 2) for 7 days after a spinal cord contusion at T9 and 1 ml blood drawn from the heart for blood cell analysis.
N = within the normal range.

C16 Reduces Monocyte Transmigration In Vitro

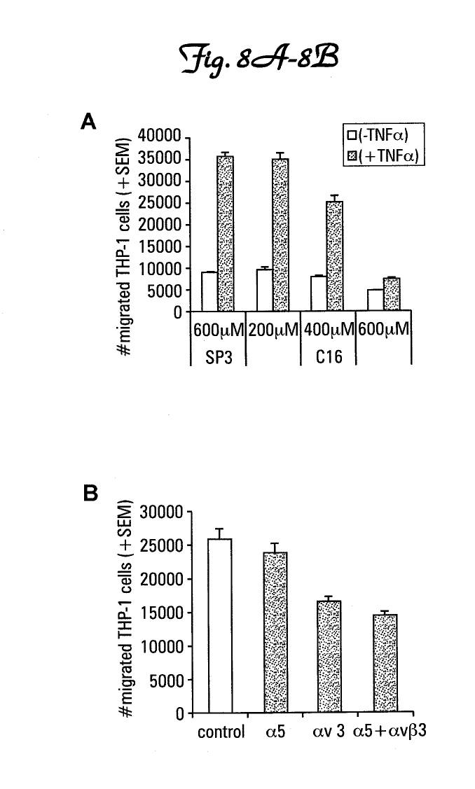

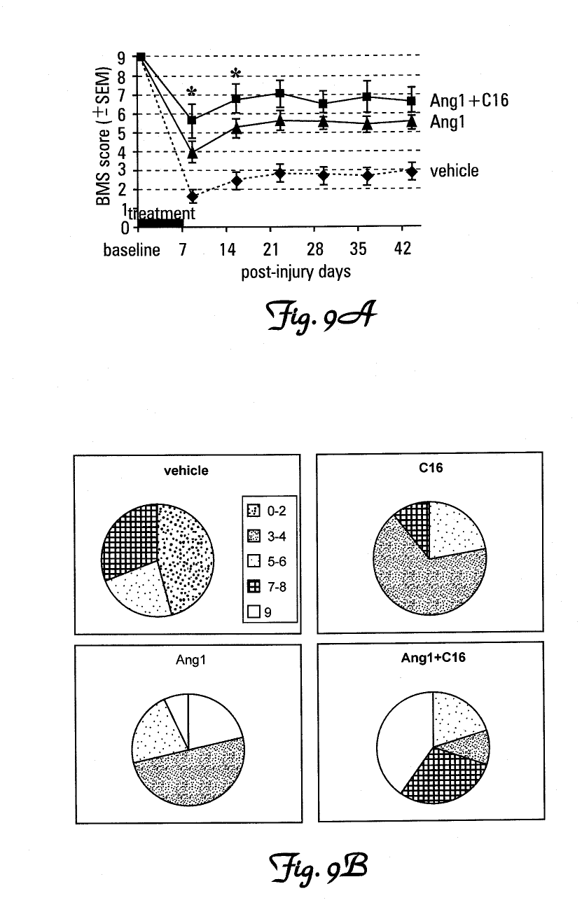

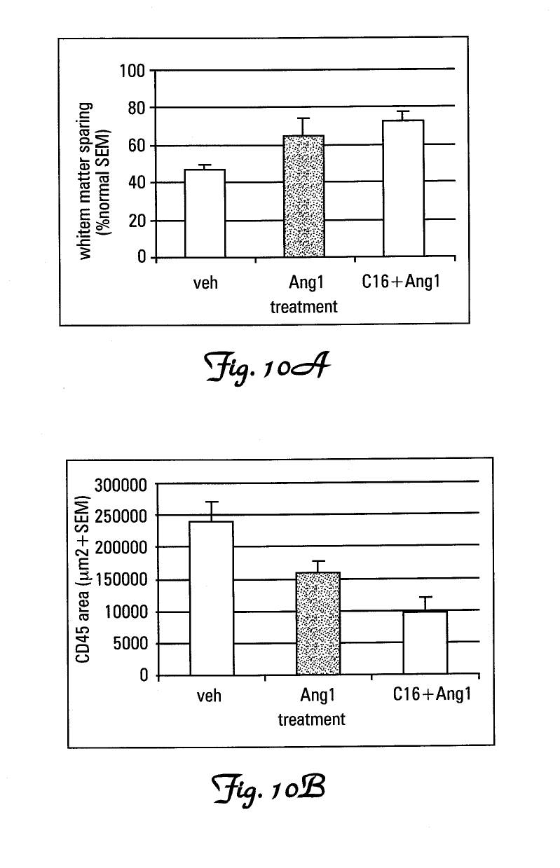

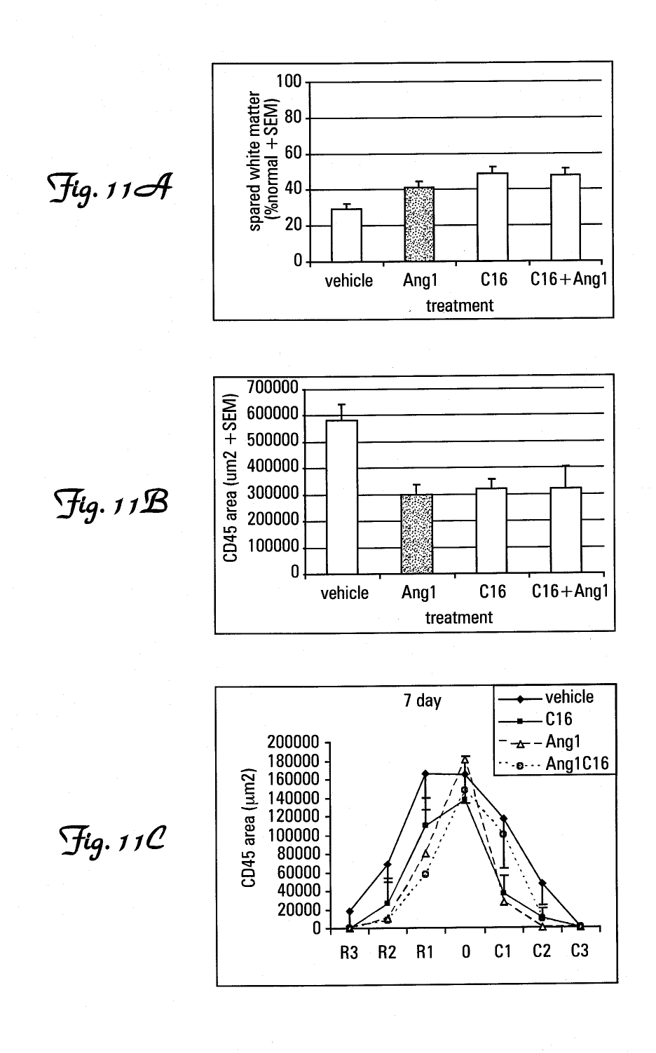

To test whether C16 could affect leukocyte extravasation, we used a transmigration assay of monocytes across an endothelial cell layer in a two-compartment culture system. Under control conditions with vehicle or up to 600 μM SP3 in the media, essentially all the monocytes crossed the endothelial barrier (FIG. 8A). C16 reduced transmigration in a dose dependent manner to 52% at 600 μM, and did so even in the presence of 15 ng/ml of the pro-inflammatory cytokine, TNFα, reducing the transmigration to 21%. To test the potential role of αvβ3 and α5β1 integrin receptors well-characterized blocking antibodies were used. Antibodies against α5 integrin did not affect transmigration and whereas αvβ3 antibodies reduced the monocyte transmigration to 64% (FIG. 8B). The more extensive blocking by C16 suggests it has additional effects that do not involve blocking the integrin receptors and are related to its agonist effects.

SUMMARY

Spinal cord injury results in loss of function and progressive secondary tissue degeneration leaving many injured people with severe neurological disabilities. There are no satisfactory neuroprotective treatments. Blood vessel loss and inflammation contribute to secondary degeneration after spinal cord injury. The potential role of αvβ3 integrin as it promotes endothelial survival and angiogenesis elsewhere was investigated. The αvβ3, but not α5β1, integrin was expressed in blood vessels in the injury epicenter following a spinal cord contusion at T9 in C57B1/6 mice. Daily intravenous injections with an αvβ3/α5β1 integrin agonist (laminin-based peptide C16; KAFDITYVRLKF (SEQ ID NO:2)) rescued white matter at the injury epicenter and improved locomotor function, compared to vehicle or an inactive peptide. These neuroprotective effects were maintained over 6 weeks by a 2-week C16 treatment started 4 hours after the injury. C16 was as effective after 7 days when injected only on day 1, suggesting that it affects acute post-injury phases. C16 rescued rescued functioning blood vessels 1 day after injury which remained present up to 6 weeks. The improved vascularity correlated only modestly with white matter sparing and improved function, suggesting that other actions also make C16 neuroprotective. C16 also reduced leukocyte extravasation and microglial/macrophage activation which peak around day 7. The reduced inflammation correlated well with the improved white matter sparing and locomotor function. In vitro, C16 reduced monocyte transmigration across endothelial cells to ˜20%. Transmigration was less reduced by αvβ3 and not by α5β1 integrin antibodies, suggesting that C16 acts through αvβ3 integrin and that the greater effects reflect C16's agonist properties. These results identify endothelial αvβ3 as an important regulator of vascular function and inflammation that can be pharmacologically activated for neuroprotection after injury to the nervous system.

### Example 2

Adult C57B1/6 mice received a contusion injury at the thoracic spine level 9. Starting 4 hours after the injury, the mice received daily intravenous injections of control vehicle, Ang-1 or Ang-1 plus C16 (all at 100 μg/day) for 7 days. Behavioral analyses over 6 weeks demonstrated that treated mice had a significant and large sparing of locomotor function compared to vehicle injected mice. Many of the treated mice had an apparently normal function. Combining data from two independent experiments demonstrated the administration of C16 with Ang-1 resulted in a score of 1-1.5 higher than with Ang-1 alone. The Ang-1C16 coadministration improved the BMS score to close to 7 vs. 5.5 with Ang-1 or C16 vs. 3 with vehicle (all on a 9 point scale, with 5 being weight-bearing and stepping, over 7 indicating highly functional locomotion). Histological analyses of the first 6 week group showed a sparing of myelin at the injury site and a reduced infiltration of CD45-positive leukocytes in both treated groups. CD45 levels were reduced more in Ang-1+C16 treated mice than in Ang-1-treated mice. Analyses of another group of mice one week after injury confirmed that the sparing of myelin and reduction of inflammation occurred mainly during the first week after injury, coinciding with the known period of peak inflammation and secondary degeneration. Analyses at 24 hours showed blood vessel protection and reduced inflammation at the spinal cord injury site by both C16 and Ang-1.

Angiopoietin-1 tetra-fibrinogen-like domain (Ang-1TFD or Ang-1^{4FD}) mimics the natural multimeric Ang-1. Ang-1TFD contains two human Ang-1 fibrinogen-like domains fused to a human Fc domain. Ang-1TFD activates the endothelial receptor tyrosine kinase Tie2 receptor, which is selectively expressed by endothelial cells, activation of which improves vascular survival and function.

Ang-1TFD, also known as Human BowAng-1 Fc, was produced by in Chinese Hamster Ovary cells. Ang-1 was provided by Regeneron Pharmaceuticals Inc. and C16 was produced commercially.

### Example 3

### Targeting Vascular Responses with Intravenous Angiopoietin-1 and αvβ3 Integrin Peptide is Neuroprotective after Spinal Cord Injury

Blood vessel loss and inflammation cause secondary degeneration following spinal cord injury. The αvβ3 integrin and angiopoietin-1 promote endothelial cell survival during developmental or tumor angiogenesis. Daily intravenous injections with an αvβ3 integrin binding peptide (C16) and/or an angiopoietin-1 mimetic following a T9 spinal cord contusion in C57B1/6 mice rescues blood vessels and white matter and reduces detrimental inflammation at the injury site. When given together, the effects resulted in almost complete recovery of function, whereas placebo-treated mice had hind-limb paralysis. Preserved vascularity and reduced inflammation correlated with improved outcomes. The treatment had lasting effects when started 4 hours following injury and terminated after one week, and had no observable adverse effects. C16 reduced leukocyte transmigration in vitro in an αvβ3 integrin-dependant manner. These results identify αvβ3 integrin and angiopoietin-1 as vascular and inflammatory regulators that can be targeted in a clinically relevant manner for neuroprotection after CNS trauma.

Currently, the only neuroprotective treatment for acute spinal cord injury (SCI) in humans is methylprednisolone, but its use is controversial. SCI, particularly the common contusive and compression types, causes progressive tissue loss in part secondary to blood vessel dysfunction and inflammation at the injury epicenter. Endothelial cells (ECs) and blood vessels are lost during the first 3 days, causing ischemia. Surviving blood vessels become leaky, initiating leukocyte infiltration, which contribute to loss of myelin and tissue. Therapies targeting inflammatory responses partially improve tissue sparing and neurological function following SCI. Blood vessel loss is followed by angiogenesis at the epicenter which is maintained up to 21 days in mice. Whether rescuing damaged blood vessels or stimulating angiogenesis would improve outcomes following SCI is unknown. The roles of αvβ3 integrin and Ang-1 in neurotrauma have not been investigated.

Intravenous C16 and Ang-1 treatments seem to target vascular mechanism(s) involved in secondary tissue damage following SCI. Ang-1 reduced loss of blood vessels at the injury site as early as 24 hours post-SCI, likely reducing ischemia and subsequent tissue loss. The number of perfused blood vessels seen at 7 or 42 days post-injury correlated with the extent of white matter sparing and the improvement in locomotor function, supporting the concept that vascular protection is a viable therapeutic strategy following CNS injuries. Ang-1 also reduced permeability at 72 hours, consistent with the reduced inflammation, potentially via Ang-1 's capacity to preserve the integrity of EC tight junctions under pathological conditions. The reduction in inflammation occurred coincident with reduced microglia/macrophage activation, also likely contributing to augmented white matter sparing at the injury epicenter. The extent of white matter sparing is directly related to locomotor function, as shown here using regression analyses.

C16 also rescued blood vessels at the injury epicenter at 24 hours following SCI. possibly by activating αvβ3 integrin to promote EC survival. C16 represents a functional laminin sequence and might mimic the basement membrane attachment necessary for normal EC survival, which is disrupted after SCI. The number of LEA-labeled blood vessels increased with C16 treatment between 24 hours and 7 days following SCI and the number of blood vessels correlated with both spared white matter and locomotor function at 7 days post-injury. Collectively, these data indicate that C16 is an αvβ3 agonist for ECs and promotes therapeutic angiogenesis. More LEA-labeled blood vessels were not observed at 7 days in the vehicle-treated mice. Angiogenesis normally occurs at that post-injury time in mice, suggesting that few new vessels are perfused.

C16 reduced monocyte transmigration across an EC layer to the same extent as blocking antibodies against αvβ3 but not α5 integrins. This indicates that C16 acted as an antagonist. Some leukocytes, including monocytes, express αvβ3 integrin, which is involved in their transmigration. Thus, C16 could have occupied the αvβ3 integrin, thus interfering with ICAM-1 binding required for transmigration. αv or β3 immunoreactivity was not observed on infiltrated cells following SCI, suggesting that monocytes alter their integrin expression when they become macrophages after entrance into the spinal cord.

The C16+Ang-1 combination treatment provided superior locomotor recovery compared to the individual agents, despite the lack of a difference in white matter sparing and inflammatory measures at 7 days or 6 weeks post-SCI. This indicates that more subtle changes at the injury site or farther away are responsible for the better functional outcomes. The number of perfused blood vessels was greater with C16 than with Ang-1 at 7 days, whereas the permeability was only reduced by Ang-1. Thus, the combination treatment may combine the two beneficial effects and that C16 targets additional signaling pathways within ECs or also affects other cell types. The C16+Ang-1 combination treatment promotes a remarkable degree of improvement during the first post-injury week and lasts after its termination.

The current results reveal novel vascular- and αvβ3 integrin-related mechanisms amenable to small peptide targeting and reveal that those mechanisms can cooperate with Ang-1. This new, clinically relevant, approach of improving function after SCI by rescuing functioning blood vessels and reducing detrimental inflammation is also relevant to other acute neurological disorders. The intravenous route is readily translatable to a clinical setting and ensures that therapeutic doses are quickly reached. When dealing with acute injuries, such as neural trauma and stroke, rapid intervention is probably most efficacious. The ability to delay the treatment by 4 hours after the injury and maintain efficacy will provide successful treatment of most patients, particularly because the intravenous treatment can be started as soon as a diagnosis of SCI is made. The data also suggest that C16 and Ang-1 treatments target very early post-injury mechanisms and document that such neuroprotective treatments may be limited to the first week and still have a maximal effect. The surprising finding that a single injection of C16 is also neuroprotective suggests that such treatments could be even shorter, thus further reducing the potential for detrimental side-effects. Lastly, Ang-1 appears to affect vascular homeostasis exclusively through the Tie2 receptor which is almost exclusively expressed in ECs, potentially making it an ideal target for pharmacological i.v. treatments.

### Results

### I.V. C16+Ang-1 Treatments Greatly Reduce Locomotor Deficits Following SCI

Adult female C57B1/6 mice received a contusion at T9 using the Infinite Horizon impactor resulting in a moderately severe injury. Daily i.v. injections of 100 μg C16 or an Ang-1 mimetic (see methods; subsequently referred to as Ang-1) over 7 days provided protection of white matter at the epicenter (p<0.01 each vs. vehicle; n=5 per group; data not shown). A 300 μg/d dose did not further improve the outcome (n=5 per group). Since white matter sparing correlates with locomotor function 100 μg/d was used for subsequent experiments (Basso et al., J. Neurotrauma, 23, 635-659 (2006); Li et al., J. Neurosurg., Spine, 4, 165-173 (2006)).

C16 was first tested alone for its effects on overground locomotion using the Basso Mouse Scale (BMS). Seven days following SCI and daily i.v. injections with C16, the BMS score was 4.3±0.3 (+standard error of the mean, SEM) compared to 1.8±0.3 with vehicle or 0.7±0.3 with SP3 control peptide (FIG. 13A). The αvβ3 and α5β1 integrins were present in perfused blood vessels at the injury epicenter 1 and 3 days following contusion in other mice (FIG. 18), indicating that they could have responded to C16 peptide.

To determine whether C16 would provide lasting benefits, contused mice received daily injections with vehicle or 100 μg C16 over 14 days, starting 4 hours post-injury. The 4 hour period was chosen because this is the time in which most human SCI cases are diagnosed and within which time i.v. treatments could start. C16-treated mice had a higher BMS score than vehicle-treated mice starting at 7 days (FIG. 13A) and continuing until week 6 (5.4±0.4 vs. 4.3±0.4). A score of 5 or higher indicates weight-bearing ability and consistent plantar stepping, thus making a substantial functional difference. To test whether C16 treatment during the chronic injury phase would further improve outcomes, injured mice were treated with vehicle or C16 over the first week, and then each group was treated with vehicle or C16 during week 5 (n=4 for each group). Mice injected with C16 during the first week had higher BMS scores than vehicle-treated mice (5.8±0.6 vs. 1.4±0.4 at 6 weeks; p<0.001; data not shown). However, the second C16 (or vehicle) treatment did not modify the BMS scores in mice injected with vehicle or C16 over the first week (p>0.1; paired t-Test).

Finally, C16 was combined with Ang-1. Mice received daily i.v. injections of vehicle, Ang-1, or C16+Ang-1 for 7 days, starting 4 hours following contusion. Two separate experiments had similar results and were combined. At 8-9 days post-injury, the BMS score was higher with Ang-1 and C16+Ang-1 than with vehicle treatment (FIG. 13B). These differences were maintained following termination of the treatment, with all groups reaching a plateau. The average score of the C16+Ang-1 group during the last two weeks was not significantly different from the Ang-1 or C16 groups. However, the BMS scale is non-linear and to get better insight into the functionality of the mice, the scores were grouped in functional categories (FIG. 13C). To the untrained eye, mice with a score of 7 and above walk normal, which occurred in 70% of the mice treated with C16+Ang-1, vs. 28% with Ang-1, 11% with C16 and 0% with vehicle. Conversely, one third of the vehicle-treated mice had hind-limb paralysis (scores 0-2), vs. none in the other groups. When comparing only mice with scores of 5 and higher (weight-bearing and stepping), the C16+Ang-1 treatment was better than any of the other treatments (FIG. 13D). These results reveal superior and lasting effects on locomotor function by i.v. injection of an αvβ3 integrin binding peptide together with the Tie2 ligand Ang-1.

### C16 and Ang-1 Reduce White Matter Loss Following SCI

The injury epicenter was determined in transverse sections by the minimum area of myelin along the spinal cord axis. In contused mice treated for 7 days with vehicle (FIG. 14B), only a portion of the ventral white matter remained present compared to sham-operated mice (FIG. 14A). With C16, much of the ventral and lateral white matter was spared (FIG. 14C). C16-treated mice had a smaller lesion volume (FIG. 19). The cross-sectional area of white matter at the epicenter was greater with C16, Ang-1 or C16+Ang-1 than with vehicle at both the 7 and 42 day post-injury times (FIG. 14D). Regression analyses showed a correlation between epicenter white matter sparing and BMS scores at 7 and 42 days. However, treatment with C16+Ang-1 did not result in more white matter than C16 or Ang-1, suggesting that white matter sparing alone did not account for the better locomotor function with C16+Ang-1. The white matter area was not significantly different between 7 and 42 days post-injury, irrespective of treatment group. Together with the substantial locomotor improvement seen as early as 7 days, this suggests that C16 and Ang-1 affect mechanism(s) such as blood vessel loss or inflammation during the acute and early sub-acute phase. This is also supported by the finding that one C16 injection (1× C16) immediately following SCI was as effective as the 7 day treatment in rescuing white matter (FIG. 14D) and locomotor function at 7 days (BMS: 3.8±0.6 vs. 4.3±0.3; p=0.26) compared to one injection of vehicle (BMS: 2.0±0.4; p<0.05).

### C16 and Ang-1 Improve Vascularity at the Injury Site

The presence of perfused blood vessels, which is relevant to tissue preservation, was assessed by injecting an EC-binding FITC-conjugated LEA lectin i.v. 30 minutes before euthanasia (FIG. 15A-C). At 24 hours, 7 days and 42 days following contusion, C16- and Ang-1-treated mice had more perfused blood vessels in a spinal cord segment including the epicenter and 1 mm rostral and caudal from it than vehicle-treated mice (FIG. 15D). At 24 hours, C16- or Ang-1-treated mice had more LEA+blood vessels (44% or 40%, both p<0.05) than vehicle-treated mice (27%; FIG. 15D), suggesting that both C16 and Ang-1 rescue a proportion of injured blood vessels. C16-treated mice had more blood vessels at 7 days than at 24 hours (p<0.01), suggesting that C16 stimulates angiogenesis in vivo as it does in vitro. This was not seen with Ang-1, consistent with its lack of angiogenic properties. At 7 and 42 days post-injury, the number of LEA+blood vessels correlated with BMS scores (FIG. 15E,G) and white matter sparing (FIG. 15F,H). Thus, improved vascularity may rescue white matter leading to improved function. However, the finding that C16-treated mice had similar BMS scores as the Ang-1 treated mice, despite greater vascularity at 7 days, suggest that increased vascularity alone is not sufficient to explain the treatment-induced improvements. The extent of correlation between blood vessels and BMS scores or white matter (R² values) was reduced from 7 and 42 days post-injury (FIG. 15E vs. G and F vs. H). This suggests that additional non-vascular mechanisms not directly related to the C16 or Ang-1 treatments (e.g., plasticity, remyelination) contribute to outcomes during the chronic post-injury phase.

### C16 and Ang-1 Reduce Inflammation Following SCI

Inflammation contributes to tissue loss after SCI. CD45 was used as a marker for extravasated leukocytes and CD68 as a marker for activation of resident microglia and extravasated macrophages. The latter are major contributors to demyelination. At 7 days post-contusion, the presence of CD45+ cells was greatly increased in vehicle-treated mice over most of the cross-section of the spinal cord at the injury epicenter (FIG. 16A) and over several mm rostral and caudal to it. This infiltration was markedly attenuated by C16 (FIG. 16B). A similar result was seen with CD68 staining (FIG. 16C,D). Sham-operated mice had essentially no immunostaining (not shown). Blood-work showed that the leukocyte numbers were within the normal range in both the vehicle and C16 groups 7 days post-injury. Some mice had low red blood cell and thrombocyte counts probably due to bleeding caused by surgery. This shows that C16 reduces extravasation into the spinal cord. Quantification of the area of immunostaining at 1 mm distances in a spinal cord segment from 3 mm rostral to 3 mm caudal from the epicenter showed that C16, Ang-1 or C16+Ang-1 treatments reduced inflammation at all post-injury times (FIG. 16). As early as 24 hours post-injury and C16- or Ang-1 treatment reduced the area of CD68 staining by ˜25% compared to vehicle (FIG. 16E,F). This suggests that targeting very early mechanisms reduces secondary pathology following SCI, including vascular dysfunction and subsequent detrimental inflammation. However, the single bolus injection of C16 (1× C16) was less effective than the 7 day treatment in reducing leukocyte infiltration (CD45) and microglia/macrophage activation (CD68) at 7 days post-injury (FIG. 16E,F). This suggests that 7 day treatments might have more lasting neuroprotective effects. Regression analyses showed that inflammation at 7 or 42 days post-injury correlated with white matter loss and reduced BMS scores. This suggests that the reduced inflammation following C16, Ang-1 and C16+Ang-1 treatments contributed to better outcomes. The effects of C16+Ang-1 on CD45 and CD68 staining was not significantly different from C16 or Ang-1, suggesting that additional mechanisms contribute to the BMS improvement with combination treatment.

### Ang-1 but not C16 Reduces Blood Vessel Leakiness Following SCI

The combination treatment of C16 and Ang-1 provided greater improved locomotor function. The data suggest that the difference is not explained by differential effects on inflammation or rescue of blood vessels, suggesting that these agents might also affect different mechanisms. C16, but not Ang-1, appears to have induced angiogenesis between day 1 and 7, possibly increasing functions of surviving white matter containing long-projecting axons. Ang-1 reduces vascular permeability under inflammatory conditions. Increased permeability contributes to tissue loss following SCI and was measured here by the amount of luciferase that extravasated into the spinal cord at the injury site after i.v. injection 20 minutes before analysis. With Ang-1 treatment, luciferase values were not different at 24 hours following SCI but were reduced to 55±13% of vehicle at 72 hours (p<0.05). C16 had no significant effect at either time point. This suggests that Ang-1 treatment reduces pathological permeability during the sub-acute phase, possibly explaining why it also improves function despite lower vascularity compared to that seen with C16 treatments.

C16 Reduces αvβ3-Dependent Monocyte Transmigration In Vitro

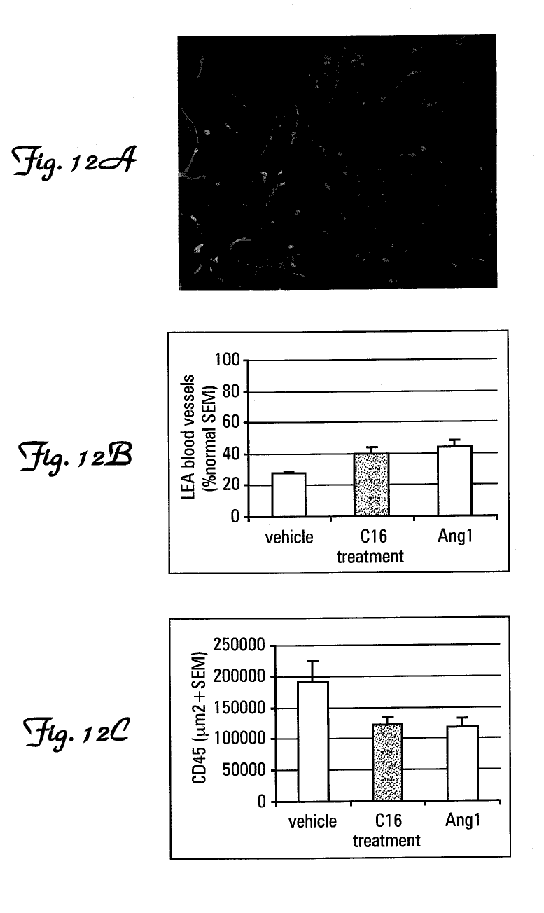

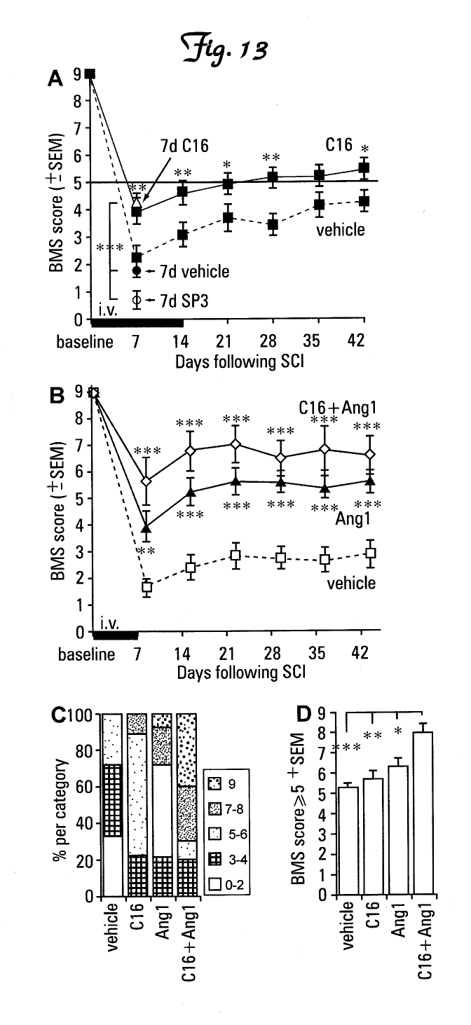

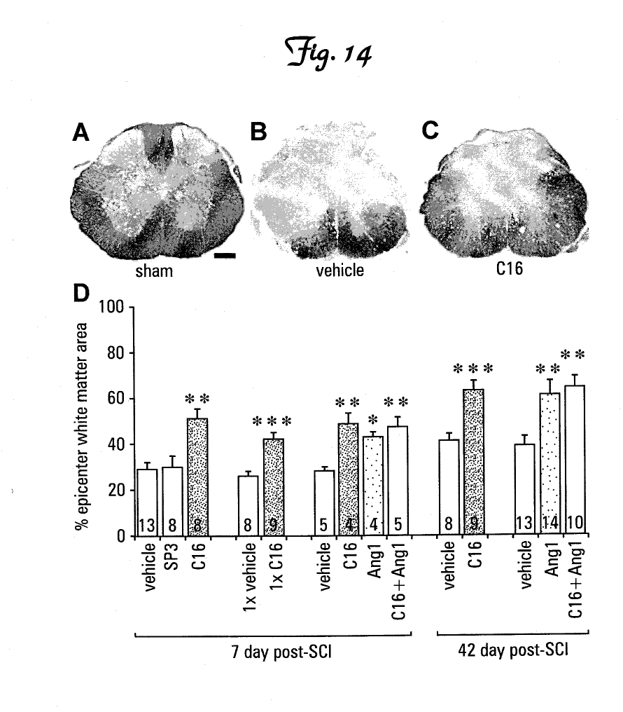

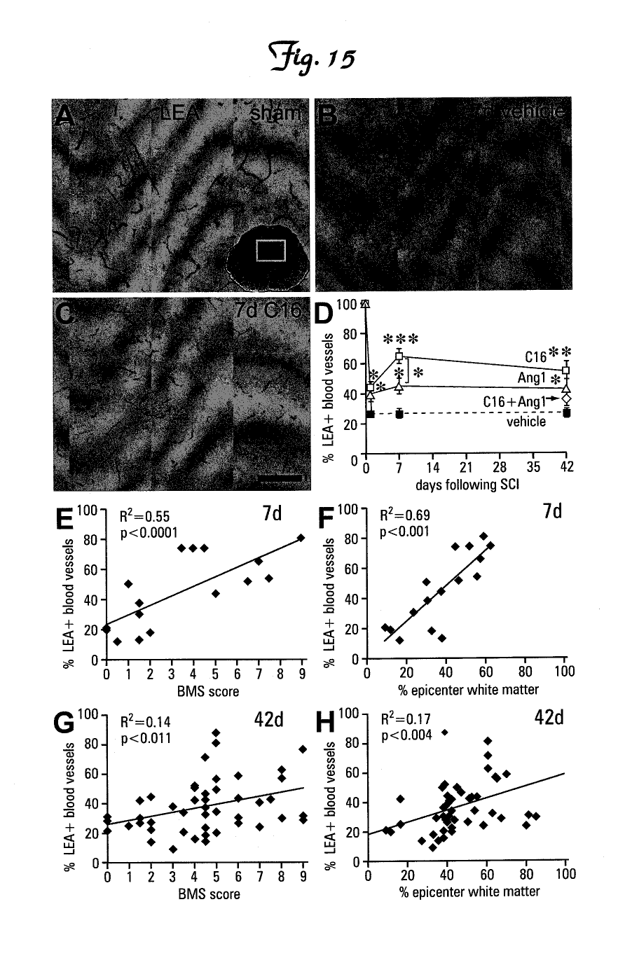

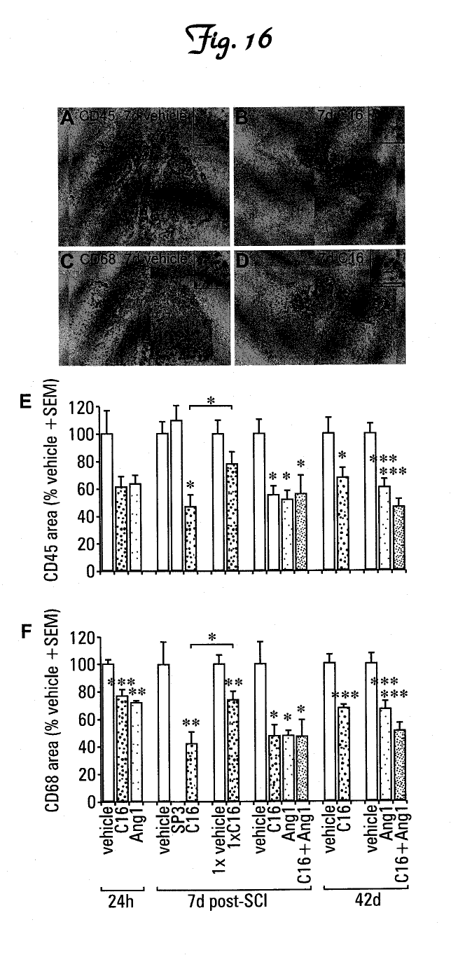

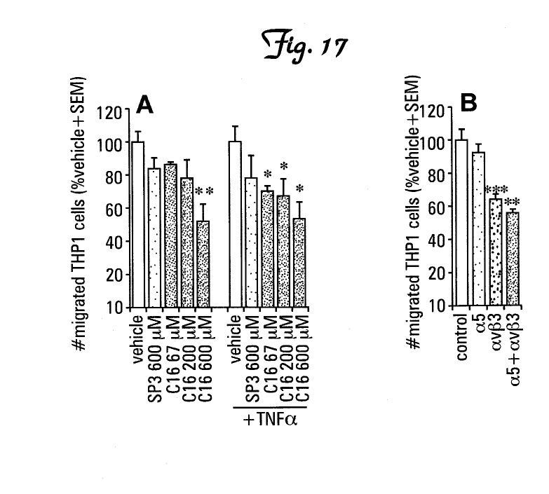

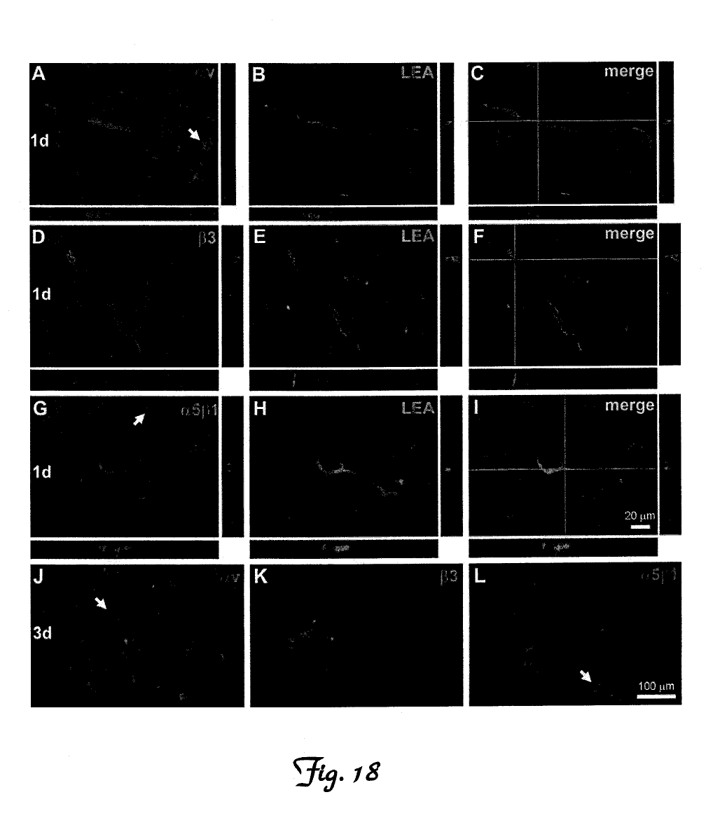

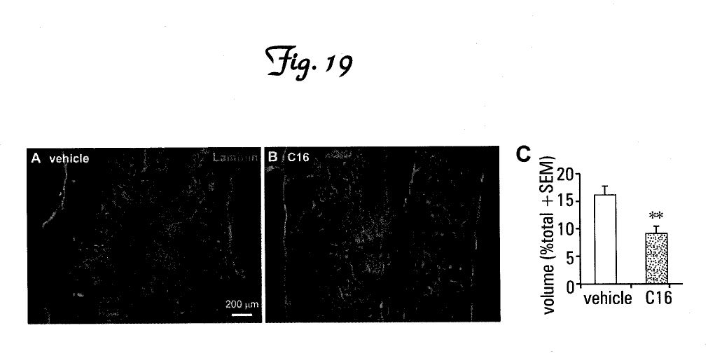

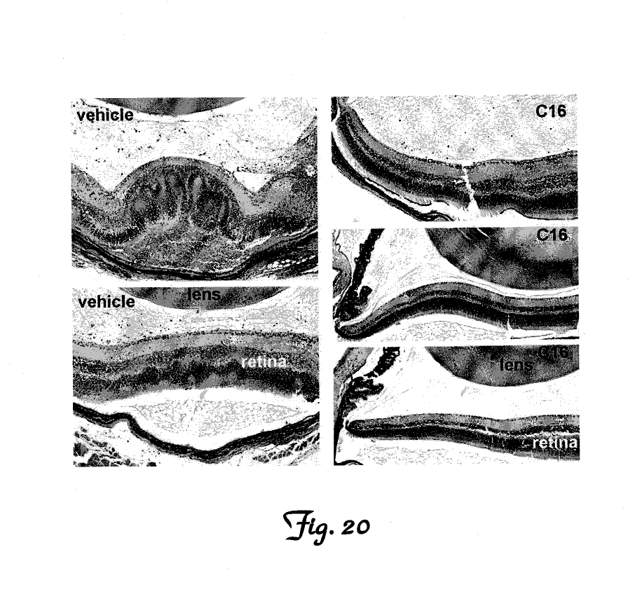

To determine the integrin target of i.v. C16 that controls inflammation, monocyte transmigration was measured across an EC layer in a two-compartment culture system. Under control conditions (vehicle or 600 μM SP3 peptide), about 5% of monocytes crossed the EC barrier. C16 reduced transmigration by 48% at 600 μM (FIG. 12A). When the ECs were stimulated with the pro-inflammatory cytokine, TNF-α (15 ng/mL), SP3 had no effect but C16 reduced C16 reduced the number of transmigrated cells already at 67 μM. Blocking antibodies against αvβ3 integrin reduced the monocyte transmigration by 36% whereas α5 integrin antibodies had no effect (FIG. 12B). This suggests that C16 reduces transmigration by blocking αvβ3 integrin, which would reduce monocyte binding to ICAM-1, an interaction important for transmigration³⁶.

### METHODS

### Animals

A total of 272 female C57BL/6 mice were used (7-11 weeks, 16-24 g at the time of SCI; Jackson Laboratory, Bar Harbor, Me.) and age- and weight-matched between groups within an experiment. All animal procedures were performed according to University of Louisville IACUC protocols and the National Institutes of Health guidelines. All invasive procedures were performed under deep anesthesia obtained by an intraperitoneal injection of per 0.4 mg/gram body weight Avertin (2,2,2-tribromoethanol in 0.02 ml of 1.25% 2-methyl-2-butanol in saline, Sigma-Aldrich, St. Louis, Mo.).

Surgeries, behavioral measurements, and quantification of histological results were done by investigators blinded to the treatments. Treatment solutions were assigned in a randomized order and were prepared and coded by someone (TH) different than the surgeon (SH). Spinal cords of individual mice were randomly coded before histological processing and un-blinded only after analyses.

### Spinal Cord Injury

The mice were anaesthetized and their backs shaved and cleansed with betadine (Purdue Products L.P., Stamford, Conn.). Lacrilube opthalamic ointment (Allergen, Irvine, Calif.) was placed on their eyes to prevent drying and 50 mg/kg of gentamicin (Boehringer Ingelheim, St. Joseph, Mo.) was administered subcutaneously to reduce infection. After a midline incision and laminectomy of the T9 vertebra, spinal cord contusions were induced using the Infinite Horizon (IH) impactor with the force set at 50 kdyn (PSI, Lexington, Ky.) (Scheff et al., J. Neurotrauma, 20, 179-193 (2003)). The vertebral column was stabilized in a frame with rigid steel clamps inserted under the transverse processes. After the injury, the muscles were closed in layers, the skin incision was closed with 7 mm metal wound clips and 2 ml of lactated Ringer's solution was given subcutaneously. Bacitracin zinc antibiotic ointment (Altana, Melville, N.Y.) was applied to the incision area. Food was placed on the bottom of the cage and water bottles with long sipping tubes were used. Buprenorphine (0.05 mg/kg) was given subcutaneously at 48 hours post-injury to reduce pain. Bladders were manually expressed twice daily until the mice had regained partial voluntary or autonomic voiding, at which time they were reduced to once a day manual expression until full voluntary or autonomic voiding was obtained. Surgeries were performed at room temperature with the mice positioned on a heating pad to maintain body temperature. After the surgery, mice were placed on fresh alpha dry bedding with cages placed on a water circulating thermal pad (37° C.) overnight before being returned to the animal care facility. Metal sutures were removed after 7 days.

### Intravenous Injections

For i.v. injection on the day of SCI, a midline incision was made in the ventral neck area and one of the jugular veins was exposed by blunt dissection and injected with 100 μl of sterile vehicle or vehicle containing sterile C16 peptide (KAFDITYVRLKF (SEQ ID NO:2)), SP3 peptide (RFSVAVSSHYPFWSR (SEQ ID NO:3)) or Ang-1 (Ang-1TFD). Ang-1TFD (Angiopoietin-1 tetra-fibrinogen-like domain or Ang-1^{4FD}) contains two human Ang-1 fibrinogen-like domains fused to a human Fc domain and mimics the natural multimeric Ang-1 and has biological activity in vivo. C16 is very selective as it was shown by affinity chromatography and immunoprecipitation to bind only to αvβ3 and α5β1 integrins and not α1, α2, α3, α6, β4, αvβ5. Moreover, C16 activity is blocked by addition of αvβ3 and α5β1 antibodies in vitro. C16-based control peptides were not used, as both scrambled C16 and reverse C16 have biological activity. Instead, SP3 peptide was used, which is an inactive scrambled form of an α6β1 binding peptide and has no known activity after SCI in rats or on monocyte transmigration in vitro. To improve solubility of the peptides, they were dissolved in distilled water with 0.3% acetic acid. Afterwards, the peptide solution was sterilized through a 0.22 μm disc filter and neutralized with NaOH. This solution was buffered by adding an equal volume of sterile PBS. The vehicle was prepared in the same manner without adding the peptide. After the jugular vein injection, the skin was closed by metal sutures. On the following 6 or 13 days, the solutions were injected via the tail vein. Ready access to the tail veins was achieved by starting injections at the caudal end of the base of the tail and into one vein. Injection sites were moved to the alternate left or right sides and increasingly more rostral on subsequent days.

To pre-label perfused blood vessels, the other jugular vein was exposed and injected with 100 μg/100 μLI FITC-conjugated Lycopersicon esculentum (tomato) agglutinin lectin (LEA, which labels perfused vasculature) 30 minutes before euthanasia. Following intravenous injection, LEA binds only to ECs and only to those of perfused blood vessels. This is a reliable method to simultaneously detect EC survival in perfused blood vessels.

To determine potential effects of C16 on the number of peripheral leukocytes, 1 ml blood was drawn from the heart just before perfusion-fixation in mice that had been treated with vehicle or C16 for 7 days. The blood was collected in EDTA-coated tubes and blood counts were performed by Drew Scientific Inc. (Oxford, Conn.).

### Functional Testing

Functional recovery after SCI was determined weekly by open-field overground locomotor performance using the BMS (Basso et al., J Neurotrauma, 23(5), 635-659 (2006)). Before each evaluation, the mice were examined carefully for perineal infections, wounds in the limbs, or tail and foot autophagia, as they could influence stepping. Such mice were excluded from the BMS test. The mice were acclimatized to the testing area for at least 25 minutes, including individually handling for at least 5 minutes, for 3 days and then tested for baseline values before surgery. The mice in the 7-day experiments were tested on the day of euthanasia and the mice in the 6 week chronic group were tested every week starting on day 8 or 9 after the contusion.

### Histological Procedures

Mice were perfused transcardially with 10 ml PBS followed by 20 ml of 4% paraformaldehyde in 0.1M phosphate buffer (pH 7.4). Afterwards, the spinal cords were carefully dissected out and 1 cm segments containing the injury site were post-fixed for 24 h in the same fixative at 4° C. and then cryoprotected at 4° C. in 30% phosphate buffered sucrose overnight. Up to 10 segments were embedded in TissueTek (Sakura Finetek, Torrance, Calif.) with their injury sites aligned. This ensures that groups within an experiment are processed for histology in the same manner. Twenty consecutive 20 μm transverse sections per 1 mm rostro-caudal distance along the spinal cord axis were cut on a cryostat and thaw-mounted onto charged microscope slides. In the set of mice used to determine effects of C16 on the volume of tissue loss, the cord was cut in the horizontal plane. The sections were stored in sequence at −20° C. until further use.

To detect myelin in white matter tracts, one of every five of the transverse sections at each rostro-caudal 1 mm level were stained with a modified eriochrome cyanine staining protocol. After thawing and drying for 1 to 2 hours in a slide warmer at 37° C., the slides were placed in xylene at room temperature for 2×30 minutes, then through graded ethanol solutions (twice each in 100 and 95% ethanol, once in 70% ethanol, and twice in ddH₂O) and stained with 0.2% eriochrome cyanine RS in 0.5% sulfuric acid and 0.4% ferric ammonium sulfate for 30 minutes. Afterwards, the slides were gently washed in running tap water for 5 minutes, and then briefly rinsed in ddH₂O. The slide was differentiated in 5% ferric ammonium sulfate for 5 to 10 minutes, briefly rinsed in ddH₂O, dehydrated briefly through graded ethanol solutions, cleared through xylene, and the sections coverslipped in Entellan (Electron Microscopy Sciences, Hatfield, Pa.). The injury epicenter was determined for each mouse by the rostro-caudal level that contained the least amount of spared myelin per transverse section. This epicenter level was used to align all the other histological measurements for each mouse. In case of the 24 hr post-injury mice, the greatest loss of LEA-labeled blood vessels was used to determine the epicenter.

Adjacent sections at each mm level were processed for double- or triple immunofluorescent staining to detect CD45 (leukocytes), CD68 (activated microglia/macrophages), PECAM1 (ECs), αv, β3 or α5β1 integrin, or in some cases laminin to define the area of tissue loss. In mice, lost tissue after SCI is replaced by a fibroblast-rich stroma, which has been identified as a heterodomain and is rich in laminin. Slides were warmed for 20 min on a slide warmer, a ring of wax applied around the sections with a PAP pen (Invitrogen, Carlsbad, Calif.), and the slides rinsed in 0.1 M Tris-buffered saline (TBS) for 10 min. After blocking non-specific staining with 10% donkey serum in TBS containing 0.3% Triton X-100 (TBST) for 1 h at room temperature, sections were incubated overnight at 4° C. in TBST containing 5% donkey serum and rat anti-CD45 (1:500, Cat# CBL1326, Chemicon International, Temecula, Calif.), rat anti-CD68 (1:1000, Cat# MCA 1957, ABD Serotec, Kindlington, Oxford, UK) or rat anti-PECAM1 (1:500, Cat# 550274, BD Pharmingen, San Jose, Calif.), rabbit anti-αv integrin (1:1000, Cat# AB1930; Chemicon), rabbit anti-β3 integrin (1:1000, Cat# AB1932, Chemicon), rat anti-α5β1 integrin (1:100, Cat# MAB1984, Chemicon), or rabbit anti-laminin IgG (1:300, Cat# L9393, Sigma). As a control, purified rat or rabbit IgG (IR-RB-IGG, IR-IB IGG, Innovative Research, NoVi, Mich.) was used at the same concentrations instead of the primary antibody. Next, the sections were incubated in TBST containing 5% donkey serum and 1:500 of appropriate secondary antibodies (donkey TRITC-conjugated Fab′ fragments, Invitrogen, Carlsbad, Calif.; Alexa594) for 1 h at room temperature. Finally, the sections were coverslipped with antifade Gel/Mount aqueous mounting media (SouthernBiotech, Birmingham, Ala.). In between steps, sections were washed 3 times 10 min in TBS. To measure the volume of tissue loss as determined by laminin staining in the horizontal sections, the sections were processed using a Vectastain ABC-DAB staining protocol according to the manufacturer's instructions (Vector Labs, Burlingame, Calif.).

### Quantitative Measurements and Statistical Analyses

Sections were examined using a Leica DMIRE2 brightfield and fluorescence microscope and images digitized with an attached Spot RTKE camera (Diagnostics Inc., Sterling Height, Mich.). EC, CD45 and CD68 staining through the entire plane of the transverse sections was digitized using a 5× objective and the area occupied by the staining calculated for the three sections per rostro-caudal level by using the threshold feature of Scion Image software (Scion Corporation, Frederick, Md.). For analysis of LEA-labeled blood vessels, images of the dorsal column and adjacent gray matter and of the ventrolateral funiculus and adjacent gray matter were taken at the injury epicenter and at 1 mm rostral and caudal to it using a 20× objective. The area of LEA was determined with Scion Image. To provide a measure of the number of blood vessels, the number of LEA-positive vessels intersecting 100 μm spaced horizontal (5) and vertical (6) lines were counted in each image. To determine the volume of the heterodomain, every 5^{th} horizontal section was stained for laminin using DAB as substrate, the heterodomain was circled in each section as well the outline of the entire 10 mm length of the spinal cord segment using Neurolucida software (MBF Bioscience, Williston, Vt.). The software calculated the volume based on the section interval and the area per section and this was expressed as a percentage of the total 10 mm segment.

### Transendothelial Migration Assay

Human aortic ECs (BioWhittaker, Walkersville, Md.) were plated on permeable filters in Transwell culture plates (Costar, Cambridge, Mass.) at 4×10⁴ cells per well. They were grown for 72-96 h to reach confluency at 37° C. in Dulbecco's modified Eagle's medium Ham's F-12 (DMEM F-12; BioWhittaker) plus EGM-2 SingleQuots™ supplement (BioWhittaker) which contains hydrocortisone, hEGF, FBS, VEGF, hFGF-B, R3-IGF-1, ascorbic acid, heparin, and gentamicin/amphotericin-B, and 10-20% fetal calf serum. Afterwards, the inner chamber of the Transwell system was loaded with 2×10⁵ monocytic THP-1 cells (American Type Culture Collection, Manassas, Va.) and preincubated in medium with or without 15 ng/ml of TNF-α (PeproTech, Rocky Hill, N.J.) for 5 hr at 37° C. C16 peptide was added into the medium of the inner chamber at 25, 50, 100, 200, 400, 600 μM, and SP3 was added at 600 μM. Peptides were centrifuged to remove any precipitates before addition. The vehicle served as the control. THP-1 cells were allowed to transmigrate for 6 h at 37° C. and the number of THP-1 cells that crossed EC layer was counted. Values were derived from 3 wells per concentration and three independent experiments were performed. To assess the functions of integrins, azide-free blocking antibodies against human αvβ3 (Cat# MAB1976Z, Chemicon; also known as LM609) and human α5 (Cat# MAB1956Z, Clone P1D6 Chemicon) integrin were added in a separate experiment without C16.

### Statistics

Statistically significant differences between two groups were determined by one-tailed t-Test if an outcome was hypothesized beforehand, two-tailed if not, and paired when an individual animal's response was compared before and after a treatment. One-way ANOVA followed by posthoc Tukey analysis was performed to compare groups of three or more. For analysis of differences in BMS scores between treatment groups over time, two way repeated measures ANOVA with post-hoc Tukey was performed. Regression analyses were performed with a confidence interval set at 95%. Tests were performed using Excel (Microsoft Office XP Professional) and Sigmastat (Systat Software Inc., San Jose, Calif.) software. A p<0.05 was considered statistically significant. Values for groups are presented as an average ± standard error of the mean (SEM).

### Example 4

### Treating Uveitis

Remarkable suppression of uveitis in the eyes of mice was demonstrated following administration of the C16 peptide (FIG. 20). Uveitis is a common and severe human condition leading to blindness. The autoimmune disease was induced in mice by injecting IRBP protein. The disease develops over 2 weeks, including severe inflammation of the retina and cloudiness of the eye as seen with fundoscopy.

Mice were treated with tail vein injections from days 2-9 after IRBP injection with C16. Two vehicle mice developed the disease, as expected. The three C16 treated mice had clear eyes throughout the two weeks as determined by fundoscopy and did not, or only mildly in one case, show inflammation in the histological sections through the eye. In an additional experiment, the C16 peptide will be administered after diagnosis of uveitis.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

1. A therapeutic method for treating nervous system injury in a mammal, comprising administering to the mammal in need of such therapy an effective amount of a therapeutic compound, wherein the therapeutic compound is a 4 to 20 amino acid peptide comprising a YVRL (SEQ ID NO:1) motif that has activity at the αvβ3 and/or α5β1 integrin receptor.

2-5. (canceled)

6. A therapeutic method for treating an inflammatory condition in a mammal, comprising administering to the mammal in need of such therapy an effective amount of a therapeutic compound, wherein the therapeutic compound is a 4 to 20 amino acid peptide comprising a YVRL (SEQ ID NO:1) motif that has activity at the αvβ3 and/or α5β1 integrin receptor.

7. A therapeutic method for treating a degenerative disorder in a mammal, comprising administering to the mammal in need of such therapy an effective amount of a therapeutic compound, wherein the therapeutic compound is a 4 to 20 amino acid peptide comprising a YVRL (SEQ ID NO:1) motif that has activity at the αvβ3 and/or α5β1 integrin receptor.

8. The method of claim 7, wherein the degenerative disorder is a neuron, axon, or myelin disorder.

9. The method of claim 7, wherein the degenerative disorder is an oligodendrocyte disorder.

10. The method of claim 7, wherein the degenerative disorder is multiple sclerosis or peripheral neuropathy.

11. The method of claim 1, wherein the therapeutic compound is administered directly into or around the injured tissue, is administered through delivery into the cerebrospinal fluid, is administered onto or directly into the eye or is administered intravenously.

12. The method of claim 1, wherein the therapeutic compound is C16.

13. The method of claim 1, wherein the compound is administered at a concentration of less than 100 mg/kg/day.

14. The method of claim 1, wherein the compound is administered at a concentration of between 1 ng/kg/day to 30 mg/kg/day.

15. The method of claim 1, wherein the compound is administered at a concentration of between 1 mg/kg/day to 10 mg/kg/day.

16. The method of claim 1, wherein the compound is administered at a concentration of between 3 mg/kg/day to 10 mg/kg/day.

17. The method of claim 1, wherein the compound is administered at a concentration of between 1 mg/kg/day to 3 mg/kg/day.

18. (canceled)

19. The method of claim 1, wherein the compound is administered for a period of about one to four weeks.

20. The method of claim 1, wherein the nervous system injury is a traumatic nervous system injury.

21. The method of claim 20, wherein the traumatic neural injury is a spinal cord injury, brain injury, or a peripheral nerve injury, an eye injury affecting the optic nerve fibers, or a skin burn.

22. The method of claim 21, wherein the traumatic neural injury is a traumatic spinal cord injury.

23. The method of claim 20, wherein the traumatic neural injury is caused by an ischemic or hemorrhagic stroke.

24. The method of claim 1, wherein the compound is administered within about 0-48 hours of injury.

25. The method of claim 1, wherein the compound is administered within about 0-24 hours of injury.

26. The method of claim 1, wherein the compound is administered within about 0-12 hours of injury.

27. The method of claim 1, wherein the compound is administered within about 0-5 hours of injury.

28. The method of claim 1, wherein the therapeutic compound is an agonist at the αvβ3 and/or α5β1 integrin receptor.

29. The method of claim 6, wherein the inflammatory condition is uveitis or Alzheimer's disease.

30. The method of claim 29, wherein the inflammatory condition is uveitis.

31. The method of claim 1, wherein the therapeutic compound is an adjuvant.

32. The method of claim 1, further comprising administering a second therapeutic compound.

33. The method of claim 32, wherein the second therapeutic compound is angiopoietin-1, or a functional analog thereof.

34. The method of claim 33, wherein the second therapeutic compound is angiopoietin-1.

35. A kit comprising a first and second therapeutic compound, wherein the first therapeutic compound is angiopoietin-1 (Ang-1), or a functional analog thereof and the second therapeutic compound is a peptide having 4 to 20 amino acids comprising an YVRL (SEQ ID NO:1) motif.

36. The kit of claim 35, wherein the second therapeutic compound is C16.

37-40. (canceled)


SEQUENCE LISTING (3 sequences)

SEQ ID NO: 1 - protein, 4 aa, Artificial Sequence
  Description of Artificial Sequence Synthetic peptide
YVRL

SEQ ID NO: 2 - protein, 12 aa, Artificial Sequence
  Description of Artificial Sequence Synthetic peptide
KAFDITYVRLKF

SEQ ID NO: 3 - protein, 15 aa, Artificial Sequence
  Description of Artificial Sequence Synthetic peptide
RFSVAVSSHYPFWSR